SEQUENCE LISTING (111 sequences)

SEQ ID NO: 1 - protein, 5 aa, synthetic construct
SSAMS

SEQ ID NO: 2 - protein, 5 aa, synthetic construct
SGLMS

SEQ ID NO: 3 - protein, 5 aa, synthetic construct
SSVMS

SEQ ID NO: 4 - protein, 17 aa, synthetic construct
GYNGGSDRAGFADSVEG

SEQ ID NO: 5 - protein, 17 aa, synthetic construct
GYNGGSDRAGYTDSVKG

SEQ ID NO: 6 - protein, 17 aa, synthetic construct
CYNGGSDRAGYTDSVKG

SEQ ID NO: 7 - protein, 12 aa, synthetic construct
YIANSGTYYWDY

SEQ ID NO: 8 - protein, 13 aa, synthetic construct
SGSSSNIGSNTVN

SEQ ID NO: 9 - protein, 13 aa, synthetic construct
SGSTSNIGHRTVN

SEQ ID NO: 10 - protein, 13 aa, synthetic construct
SGSSSNIGNNAVN

SEQ ID NO: 11 - protein, 13 aa, synthetic construct
SGSSSNIGSNIVN

SEQ ID NO: 12 - protein, 13 aa, synthetic construct
SGGISNIGSNIVN

SEQ ID NO: 13 - protein, 14 aa, synthetic construct
TGSSSNIGAGYDVH

SEQ ID NO: 14 - protein, 13 aa, synthetic construct
SGSISNIGSNTVN

SEQ ID NO: 15 - protein, 13 aa, synthetic construct
SGTRSNIGTNTVN

SEQ ID NO: 16 - protein, 7 aa, synthetic construct
NDSQRPS

SEQ ID NO: 17 - protein, 7 aa, synthetic construct
RNNQRPS

SEQ ID NO: 18 - protein, 7 aa, synthetic construct
KNNQRPS

SEQ ID NO: 19 - protein, 7 aa, synthetic construct
SNNQRPS

SEQ ID NO: 20 - protein, 7 aa, synthetic construct
SGNQRPS

SEQ ID NO: 21 - protein, 7 aa, synthetic construct
STNQRPS

SEQ ID NO: 22 - protein, 7 aa, synthetic construct
GNTNRPS

SEQ ID NO: 23 - protein, 7 aa, synthetic construct
NHNQRPS

SEQ ID NO: 24 - protein, 7 aa, synthetic construct
SDNQRPS

SEQ ID NO: 25 - protein, 11 aa, synthetic construct
AAWDGSLNVWM

SEQ ID NO: 26 - protein, 11 aa, synthetic construct
AAWDDSLTAWV

SEQ ID NO: 27 - protein, 11 aa, synthetic construct
AAWDGSLNAWM

SEQ ID NO: 28 - protein, 11 aa, synthetic construct
SAWDHSLNAWV

SEQ ID NO: 29 - protein, 11 aa, synthetic construct
ASWDDSLNGWV

SEQ ID NO: 30 - protein, 11 aa, synthetic construct
SSWDSDLRNWV

SEQ ID NO: 31 - protein, 11 aa, synthetic construct
AAWDDRVSAWL

SEQ ID NO: 32 - protein, 11 aa, synthetic construct
AAWDDSLNGWV

SEQ ID NO: 33 - protein, 11 aa, synthetic construct
ATWDDSLSAWL

SEQ ID NO: 34 - protein, 11 aa, synthetic construct
AAWDGSLNAWL

SEQ ID NO: 35 - protein, 11 aa, synthetic construct
AVWDDRVSAWL

SEQ ID NO: 36 - protein, 121 aa, synthetic construct
EVQLVQSGGGLVQPGGSLRLSCAVSGFTLSSSAMSWVRQAPGKGFEWVSGYNGGSDRAGFADSVEGRFTISRDNAKNTLYLQMNSLKPEDTAVYYCARYIANSGTYYWDYWGQGTLVTVSS

SEQ ID NO: 37 - protein, 121 aa, synthetic construct
EVQLVQSGGGLVQPGGSLRLSCSASGFTFSSGLMSWVRQAPGKGFEWVSGYNGGSDRAGYTDSVKGRFTISRDNAKNTLYLQMNSLKPEDTAVYYCARYIANSGTYYWDYWGQGTLVTVSS

SEQ ID NO: 38 - protein, 121 aa, synthetic construct
EVQLVQSGGGLVQPGGSLRLSCAVSGFTFSSSVMSWVRQAPGKGFEWVSGYNGGSDRAGFADSVEGRFTISRDNAKNTLYLQMNSLKPEDTAVYYCARYIANSGTYYWDYWGQGTLVTVSS

SEQ ID NO: 39 - protein, 121 aa, synthetic construct
QVQLVQSGGGLVQPGGSLRLSCAASGFTFSSGLMSWVRQAPGKGFEWVSGYNGGSDRAGFADSVEGRFTISRDNAKNTLYLQMNSLKPEDTAVYYCARYIANSGTYYWDYWGQGTLVTVSS

SEQ ID NO: 40 - protein, 121 aa, synthetic construct
EVQLVQSGGGLVQSGGSLRVSCAASGFTFSSGLMSWVRQAPGKGFEWVSGYNGGSDRAGYTDSVKGRFTISRDNAKNTLYLQMNSLKPEDTAVYYCARYIANSGTYYWDYWGQGTTVTVSS

SEQ ID NO: 41 - protein, 121 aa, synthetic construct
EVQLVQSGGGLVQSGGSLRVSCAASGFTFSSGLMSWVRQAPGKGFEWVSCYNGGSDRAGYTDSVKGRFTISRDNAKNTLYLQMNSLKPEDTAVYYCARYIANSGTYYWDYWGQGTTVTVSS

SEQ ID NO: 42 - protein, 121 aa, synthetic construct
EVQLVQSGGGLVQPGGSLRVSCAASGFSFSSGLMSWVRQAPGKGFEWVSGYNGGSDRAGFADSVEGRFTISRDNAKNTLYLQMNSLKPEDTAVYYCARYIANSGTYYWDYWGQGTLVTVSS

SEQ ID NO: 43 - protein, 120 aa, synthetic construct
EVQLVQSGGGLVQPGGSLRLSCAASGFTFSSGLMSWVRQAPGGFEWVSGYNGGSDRAGFADSVEGRFTISRDNAKNTLYLQMNSLKPEDTAVYYCARYIANSGTYYWDYWGQGTTVTVSS

SEQ ID NO: 44 - protein, 110 aa, synthetic construct
QPVLTQPPSASGTPGQRVTISCSGSSSNIGSNTVNWYQQLPGTAPKLLIYNDSQRPSGVPDQFSGSKSGTSASLAISGLQSDNEADYYCAAWDGSLNVWMFGGGTKVTVL

SEQ ID NO: 45 - protein, 110 aa, synthetic construct
QAVLTQPPSVSGTPGQRVTISCSGSTSNIGHRTVNWYQQFPGAAPKLLIYRNNQRPSGVPARFSGSKSGTSASLAISGLQSEDEADYHCAAWDDSLTAWVFGGGTQLTVL

SEQ ID NO: 46 - protein, 110 aa, synthetic construct
QPVLTQPPSVSEAPRQRVTISCSGSSSNIGNNAVNWYQQLPGKAPKLLIFKNNQRPSGVPDRFSGSKSGTSASLVISGLQSEDEADYYCAAWDGSLNAWMFGGGTKLTVL

SEQ ID NO: 47 - protein, 110 aa, synthetic construct
QSVLTQPPSASGTPGQRVTISCSGSSSNIGSNIVNWYQQFPGTAPKLLIFSNNQRPSGVPDRLSGSKSGTSASLAISGLQSEDEADYYCSAWDHSLNAWVFGGGTKLTVL

SEQ ID NO: 48 - protein, 110 aa, synthetic construct
QAVLTQPPSASGTPGQRVSISCSGGISNIGSNIVNWYQHLPGTAPKLLIYSGNQRPSGVPDRFSGSKSGTSASLAISGLQSEDEADYYCASWDDSLNGWVFGGGTKLTVL

SEQ ID NO: 49 - protein, 110 aa, synthetic construct
QAVLTQPPSASGTPGQRVSISCSGGISNIGSNIVNWYQHLPGTAPKLLIYSGNQRPSGVPDRFSGSKSGTSASLAISGLQSEDEADYYCSSWDSDLRNWVFGGGTKLTVL

SEQ ID NO: 50 - protein, 110 aa, synthetic construct
QPVLTQPPSVSEAPRQRVTISCSGSSSNIGNNAVNWYQQLPGKAPKLLIYSTNQRPSGVPDRFSGSKSGTSASLAISGLQSEDEADYYCAAWDDRVSAWLFGGGTKLTVL

SEQ ID NO: 51 - protein, 111 aa, synthetic construct
QSVLTQPPSVSGAPGQRVTISCTGSSSNIGAGYDVHWYQQLPGTAPKLVIYGNTNRPSGVPDRFSGSKSGTSASLAISGLQSEDEADYYCAAWDDSLNGWVFGGGTKLTVL

SEQ ID NO: 52 - protein, 110 aa, synthetic construct
QSALTQPPSASGTPGQRVTISCSGSISNIGSNTVNWYQHLPGTAPKLLIYNHNQRPSGVPDRFSGSTSGTSASLAISGLQSEDEADYFCATWDDSLSAWLFGGGTKLTVL

SEQ ID NO: 53 - protein, 110 aa, synthetic construct
QSVLTQPPSASGTPGQRVTISCSGSSSNIGSNTVNWYQQLPGTAPKLLIYSDNQRPSGVPERFSGSKSGTSASLAISGLLSEDEADYYCAAWDGSLNAWLFGGGTKVTVL

SEQ ID NO: 54 - protein, 110 aa, synthetic construct
SYVLTQPPSASGTPGQRVTISCSGTRSNIGTNTVNWYQQLPGTAPKLLIYRNNQRPSGVPDRFSGSKSGTSASLAISGLQSEDEADYYCAVWDDRVSAWLFGGGTKLTVL

SEQ ID NO: 55 - protein, 451 aa, synthetic construct
EVQLVQSGGGLVQPGGSLRLSCAVSGFTLSSSAMSWVRQAPGKGFEWVSGYNGGSDRAGFADSVEGRFTISRDNAKNTLYLQMNSLKPEDTAVYYCARYIANSGTYYWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 451 aa, synthetic construct
EVQLVQSGGGLVQPGGSLRLSCSASGFTFSSGLMSWVRQAPGKGFEWVSGYNGGSDRAGYTDSVKGRFTISRDNAKNTLYLQMNSLKPEDTAVYYCARYIANSGTYYWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - protein, 451 aa, synthetic construct
EVQLVQSGGGLVQPGGSLRLSCAVSGFTFSSSVMSWVRQAPGKGFEWVSGYNGGSDRAGFADSVEGRFTISRDNAKNTLYLQMNSLKPEDTAVYYCARYIANSGTYYWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 451 aa, synthetic construct
QVQLVQSGGGLVQPGGSLRLSCAASGFTFSSGLMSWVRQAPGKGFEWVSGYNGGSDRAGFADSVEGRFTISRDNAKNTLYLQMNSLKPEDTAVYYCARYIANSGTYYWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - protein, 451 aa, synthetic construct
EVQLVQSGGGLVQSGGSLRVSCAASGFTFSSGLMSWVRQAPGKGFEWVSGYNGGSDRAGYTDSVKGRFTISRDNAKNTLYLQMNSLKPEDTAVYYCARYIANSGTYYWDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 451 aa, synthetic construct
EVQLVQSGGGLVQSGGSLRVSCAASGFTFSSGLMSWVRQAPGKGFEWVSCYNGGSDRAGYTDSVKGRFTISRDNAKNTLYLQMNSLKPEDTAVYYCARYIANSGTYYWDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 451 aa, synthetic construct
EVQLVQSGGGLVQPGGSLRVSCAASGFSFSSGLMSWVRQAPGKGFEWVSGYNGGSDRAGFADSVEGRFTISRDNAKNTLYLQMNSLKPEDTAVYYCARYIANSGTYYWDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 450 aa, synthetic construct
EVQLVQSGGGLVQPGGSLRLSCAASGFTFSSGLMSWVRQAPGGFEWVSGYNGGSDRAGFADSVEGRFTISRDNAKNTLYLQMNSLKPEDTAVYYCARYIANSGTYYWDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 217 aa, synthetic construct
QPVLTQPPSASGTPGQRVTISCSGSSSNIGSNTVNWYQQLPGTAPKLLIYNDSQRPSGVPDQFSGSKSGTSASLAISGLQSDNEADYYCAAWDGSLNVWMFGGGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 217 aa, synthetic construct
QAVLTQPPSVSGTPGQRVTISCSGSTSNIGHRTVNWYQQFPGAAPKLLIYRNNQRPSGVPARFSGSKSGTSASLAISGLQSEDEADYHCAAWDDSLTAWVFGGGTQLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 217 aa, synthetic construct
QPVLTQPPSVSEAPRQRVTISCSGSSSNIGNNAVNWYQQLPGKAPKLLIFKNNQRPSGVPDRFSGSKSGTSASLVISGLQSEDEADYYCAAWDGSLNAWMFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 217 aa, synthetic construct
QSVLTQPPSASGTPGQRVTISCSGSSSNIGSNIVNWYQQFPGTAPKLLIFSNNQRPSGVPDRLSGSKSGTSASLAISGLQSEDEADYYCSAWDHSLNAWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 217 aa, synthetic construct
QAVLTQPPSASGTPGQRVSISCSGGISNIGSNIVNWYQHLPGTAPKLLIYSGNQRPSGVPDRFSGSKSGTSASLAISGLQSEDEADYYCASWDDSLNGWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 217 aa, synthetic construct
QAVLTQPPSASGTPGQRVSISCSGGISNIGSNIVNWYQHLPGTAPKLLIYSGNQRPSGVPDRFSGSKSGTSASLAISGLQSEDEADYYCSSWDSDLRNWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 217 aa, synthetic construct
QPVLTQPPSVSEAPRQRVTISCSGSSSNIGNNAVNWYQQLPGKAPKLLIYSTNQRPSGVPDRFSGSKSGTSASLAISGLQSEDEADYYCAAWDDRVSAWLFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 218 aa, synthetic construct
QSVLTQPPSVSGAPGQRVTISCTGSSSNIGAGYDVHWYQQLPGTAPKLVIYGNTNRPSGVPDRFSGSKSGTSASLAISGLQSEDEADYYCAAWDDSLNGWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 217 aa, synthetic construct
QSALTQPPSASGTPGQRVTISCSGSISNIGSNTVNWYQHLPGTAPKLLIYNHNQRPSGVPDRFSGSTSGTSASLAISGLQSEDEADYFCATWDDSLSAWLFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 72 - protein, 217 aa, synthetic construct
QSVLTQPPSASGTPGQRVTISCSGSSSNIGSNTVNWYQQLPGTAPKLLIYSDNQRPSGVPERFSGSKSGTSASLAISGLLSEDEADYYCAAWDGSLNAWLFGGGTKVTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 217 aa, synthetic construct
SYVLTQPPSASGTPGQRVTISCSGTRSNIGTNTVNWYQQLPGTAPKLLIYRNNQRPSGVPDRFSGSKSGTSASLAISGLQSEDEADYYCAVWDDRVSAWLFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - DNA, 363 bp, synthetic construct
  source 1..363: mol_type = other DNA
gaagtgcagctggtgcagtctgggggaggcttggtgcagcctggggggtctctgagactctcctgtgcagtctctggattcaccttgagtagcagtgccatgagctgggtccgccaggctccaggaaaggggttcgagtgggtctcaggctataatggtggtagtgatagggcaggctttgcagactccgtggagggccgattcaccatctccagagacaacgccaagaacacgctgtatctgcaaatgaacagcctgaaacctgaggacacggccgtgtattactgtgcaagatatatagcgaatagcggtacttactactgggactactggggccaggggaccctggtcaccgtgtcgagt

SEQ ID NO: 75 - DNA, 363 bp, synthetic construct
  source 1..363: mol_type = other DNA
gaagtgcagctggtgcagtctgggggaggcttggtgcagcctggggggtctctgagactctcctgttcagcatctggattcaccttcagtagcggtctcatgagctgggtccgccaggctccaggaaaggggttcgagtgggtctcaggctataatggtggtagtgatagggcaggctatacagactccgtgaagggccgattcaccatctccagagacaacgccaagaacacgctgtatctgcaaatgaacagcctgaaacctgaggacacggccgtgtattactgtgcaagatatatagcaaatagcggtacttactactgggactactggggccaggggaccctggtcaccgtgtcgagt

SEQ ID NO: 76 - DNA, 363 bp, synthetic construct
  source 1..363: mol_type = other DNA
gaggtgcagctggtgcagtctgggggaggcttggtgcagcctggggggtctctgagactctcctgtgcagtctctggattcaccttcagtagcagtgtcatgagctgggtccgccaggctccaggaaaggggttcgagtgggtctcaggctataatggtggtagtgatagggcaggctttgcagactccgtggagggccgattcaccatctccagagacaacgccaagaacacgctgtatctgcaaatgaacagcctgaaacctgaggacacggccgtgtattactgtgcaagatatatagcaaatagcggtacttactactgggactactggggccaggggaccctggtcaccgtgtcgagt

SEQ ID NO: 77 - DNA, 363 bp, synthetic construct
  source 1..363: mol_type = other DNA
caggtgcagctggtgcagtctgggggaggcttggtgcagcctggggggtctctgagactctcctgtgcagcctctggattcaccttcagtagcggtctcatgagctgggtccgccaggctccaggaaaggggttcgagtgggtctcaggctataatggtggtagtgatagggcaggctttgcagactccgtggagggccgattcaccatctccagagacaacgccaagaacacgctgtatctgcaaatgaacagcctgaaacctgaggacacggccgtgtattactgtgcaagatatatagcaaatagcggtacttactactgggactactggggccaggggaccctggtcaccgtgtcgagt

SEQ ID NO: 78 - DNA, 363 bp, synthetic construct
  source 1..363: mol_type = other DNA
gaagtgcagctggtgcagtctgggggagggttggtgcagtctggggggtctctgagagtctcctgtgcagcctctggattcaccttcagtagcggtctcatgagctgggtccgccaggctccaggaaaggggttcgagtgggtctcaggctataatggtggtagtgatagggcaggctacacagactccgtgaagggccgattcaccatctccagagacaacgccaagaacacgctgtatctgcaaatgaacagcctgaaacctgaggacacggccgtgtattactgtgcaagatatatagcgaatagcggtacttactactgggactactggggccaggggaccacggtcaccgtctcgagt

SEQ ID NO: 79 - DNA, 363 bp, synthetic construct
  source 1..363: mol_type = other DNA
gaagtgcagctggtgcagtctgggggagggttggtgcagtctggggggtctctgagagtctcctgtgcagcctctggattcaccttcagtagcggtctcatgagctgggtccgccaggctccaggaaaggggttcgagtgggtctcatgctataatggtggtagtgatagggcaggctacacagactccgtgaagggccgattcaccatctccagagacaacgccaagaacacgctgtatctgcaaatgaacagcctgaaacctgaggacacggccgtgtattactgtgcaagatatatagcgaatagcggtacttactactgggactactggggccaggggaccacggtcaccgtctcgagt

SEQ ID NO: 80 - DNA, 363 bp, synthetic construct
  source 1..363: mol_type = other DNA
gaggtgcagctggtgcagtctgggggaggcttggtgcagcctggggggtctctgagagtctcctgtgcagcctctggattcagcttcagtagcggtctcatgagctgggtccgccaggctccaggaaaggggttcgagtgggtctcaggctataatggtggtagtgatagggcaggctttgcagactccgtggagggccgattcaccatctccagagacaacgccaagaacacgctgtatctgcaaatgaacagcctgaaacctgaggacacggccgtgtattactgtgcaagatatatagcaaatagcggtacttactactgggactactggggccaggggaccctggtcaccgtctcgagt

SEQ ID NO: 81 - DNA, 363 bp, synthetic construct
  source 1..363: mol_type = other DNA
gaggtgcagctggtgcagtctgggggaggcttggtgcagcctggggggtctctgagactctcctgtgcagcctctggattcaccttcagtagcggtctcatgagctgggtccgccaggctccaggaaaggggttcgagtgggtctcaggctataatggtggtagtgatagggcaggctttgcagacgccgtggagggccgattcaccatctccagagacaacgccaagaacacgctgtatctgcaaatgaacagcctgaaacctgaggacacggccgtgtattactgtgcaagatatatagcaaatagcggtacttactactgggactactggggccaggggaccacggtcaccgtctcgagt

SEQ ID NO: 82 - DNA, 330 bp, synthetic construct
  source 1..330: mol_type = other DNA
cagcctgtgctgactcagccaccctcagcgtctgggacccccgggcagagggtcaccatctcttgttctggaagcagctccaatatcgggagtaatactgtcaattggtatcagcaactcccaggaacggcccccaaactcctcatctataatgatagtcagcggccctcaggggtccctgaccaattctctgggtccaagtctggcacctcagcctccctggccatcagtgggctccagtctgacaatgaggctgattattactgtgcagcatgggatggcagcctgaatgtctggatgttcggcggagggaccaaggtcaccgtccta

SEQ ID NO: 83 - DNA, 330 bp, synthetic construct
  source 1..330: mol_type = other DNA
caggctgtgctgactcagcccccctcagtgtctgggacccccggacagagggtcaccatctcttgttctggaagcacctccaacatcggacataggactgtaaactggtatcagcaatttccaggagcggcccccaaactcctcatctataggaataatcagcggccctcaggggtccctgcccgattctctggctccaagtctggcacctcagcctccctggccatcagtgggctccagtctgaggatgaggctgattatcactgtgcagcatgggatgacagcctgactgcttgggtgttcggcggagggacccagctcaccgtttta

SEQ ID NO: 84 - DNA, 330 bp, synthetic construct
  source 1..330: mol_type = other DNA
cagcctgtgctgactcagccaccctcggtgtctgaagcccccaggcagagggtcaccatctcctgttctggaagcagctccaacatcggaaataatgctgtaaactggtaccagcagctcccaggaaaggctcccaaactcctcatctttaagaataatcagcggccctcaggggtccctgaccggttctctggctccaagtctggcacctcagcctccctggtcatcagtgggctccagtctgaggatgaggctgattactactgtgcagcatgggatggcagcctgaatgcttggatgttcggcggggggaccaagctgaccgtccta

SEQ ID NO: 85 - DNA, 330 bp, synthetic construct
  source 1..330: mol_type = other DNA
cagtctgtgctgactcagccaccctcagcgtctgggacccccgggcagagggtcaccatctcttgttctggaagcagctccaacatcggaagtaatattgtaaactggtaccagcagttcccaggaacggcccccaaactcctcatttttagtaataatcagcggccctcaggggtccctgaccgattatctggctccaagtctggcacctcagcctccctggccatcagtgggctccagtctgaggatgaggctgattattactgttcagcatgggatcacagcctgaatgcctgggtgttcggcggagggaccaagctgaccgtccta

SEQ ID NO: 86 - DNA, 330 bp, synthetic construct
  source 1..330: mol_type = other DNA
caggctgtgctgactcagccaccctcagcgtctgggacccccgggcagagggtcagcatctcttgttctggaggcatctccaacatcggaagtaatattgtaaactggtaccaacacctcccaggaacggcccccaaactcctcatatatagtggtaatcaacggccctcaggagtccctgaccgattctctggctccaagtctggcacctcagcctccctggccatcagtgggctccagtctgaggatgaggctgattattactgtgcatcgtgggatgacagcctgaatggctgggtattcggcggagggaccaagctgaccgtccta

SEQ ID NO: 87 - DNA, 330 bp, synthetic construct
  source 1..330: mol_type = other DNA
caggctgtgctgactcagccaccctcagcgtctgggacccccgggcagagggtcagcatctcttgttctggaggcatctccaacatcggaagtaatattgtaaactggtaccaacacctcccaggaacggcccccaaactcctcatatatagtggtaatcaacggccctcaggagtccctgaccgattctctggctccaagtctggcacctcagcctccctggccatcagtggactccagtccgaggatgaggctgactattactgctcatcatgggacagcgacctcaggaattgggtgttcggcggggggaccaagctgaccgtccta

SEQ ID NO: 88 - DNA, 330 bp, synthetic construct
  source 1..330: mol_type = other DNA
cagcctgtgctgactcagccaccctcggtgtctgaagcccccaggcagagggtcaccatctcctgttctggaagcagctccaacatcggaaataatgctgtaaactggtaccagcagctcccaggaaaggctcccaaactcctcatctatagtactaatcagcggccctcaggggtccctgaccgattctctggttccaagtctggcacctcagcctccctggccatcagtgggctccagtctgaggatgaggctgattattactgtgcagcatgggatgacagagtgagtgcttggctgtttggcggagggaccaagctgaccgtccta

SEQ ID NO: 89 - DNA, 333 bp, synthetic construct
  source 1..333: mol_type = other DNA
cagtctgtgttgacgcagccgccctcagtgtctggggccccagggcagagggtcaccatctcctgcactgggagcagctccaacatcggggcaggttatgatgtacactggtaccagcaacttccaggaacagcccccaaactcgtcatctatggtaacaccaatcggccctcaggggtccctgaccgattctctggctccaagtctggcacctcagcctccctggccatcagtgggctccagtctgaggatgaggctgattattactgtgcagcatgggatgacagcctgaatggttgggtattcggcggaggcaccaagctgaccgtccta

SEQ ID NO: 90 - DNA, 330 bp, synthetic construct
  source 1..330: mol_type = other DNA
cagtctgccctgactcagccaccctcagcgtctgggacccccgggcagagggtcaccatctcttgttctggaagcatctccaacatcggaagtaatactgtaaactggtaccagcatctcccaggaacggcccccaaactcctcatctataatcataatcagcggccctcaggggtccctgaccgattctctggctccacgtctggcacgtcagcctccctggccatcagtgggctccagtctgaggatgaggctgattatttctgtgcaacatgggatgacagcctgagtgcttggctgttcggcggagggaccaagctgaccgtccta

SEQ ID NO: 91 - DNA, 330 bp, synthetic construct
  source 1..330: mol_type = other DNA
cagtctgtgctgacgcagccgccctcagcgtctgggacccccgggcagagggtcaccatctcttgttctggaagcagctccaacatcggaagtaatactgtgaactggtaccagcagctcccaggaacggcccccaaactcctcatctatagtgataatcagcggccctcaggtgtccctgaacgcttctctggctccaagtctggcacctcagcctccctggccatcagtggcctcctgtctgaggatgaggctgattattactgtgcagcatgggatggcagcctgaatgcttggctattcggcggtgggaccaaggtcaccgtccta

SEQ ID NO: 92 - DNA, 330 bp, synthetic construct
  source 1..330: mol_type = other DNA
tcctatgtgctgacacagccaccctcagcgtctgggacccccggacagagggtcaccatctcttgttctgggaccaggtccaacatcggaactaatactgttaactggtaccagcagctcccaggaacggcccccaaactcctcatctatagaaataatcagcggccctcaggggtccctgaccgattctctggctccaagtctggcacctcagcctccctggccatcagtgggctccagtctgaggatgaggctgattactactgtgcagtatgggatgacagagtgagtgcttggctgtttggcggagggaccaagctgaccgtccta

SEQ ID NO: 93 - DNA, 1353 bp, synthetic construct
  source 1..1353: mol_type = other DNA
gaagtgcagctggtgcagtctgggggaggcttggtgcagcctggggggtctctgagactctcctgtgcagtctctggattcaccttgagtagcagtgccatgagctgggtccgccaggctccaggaaaggggttcgagtgggtctcaggctataatggtggtagtgatagggcaggctttgcagactccgtggagggccgattcaccatctccagagacaacgccaagaacacgctgtatctgcaaatgaacagcctgaaacctgaggacacggccgtgtattactgtgcaagatatatagcgaatagcggtacttactactgggactactggggccaggggaccctggtcaccgtgtcgagtgctagcaccaagggcccatcggtcttccccctggcaccctcctccaagagcacctctgggggcacagcggccctgggctgcctggtcaaggactacttccccgaacccgtgaccgtgtcgtggaactcaggcgccctgaccagcggcgtgcacaccttcccagctgtccttcagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 94 - DNA, 1353 bp, synthetic construct
  source 1..1353: mol_type = other DNA
gaagtgcagctggtgcagtctgggggaggcttggtgcagcctggggggtctctgagactctcctgttcagcatctggattcaccttcagtagcggtctcatgagctgggtccgccaggctccaggaaaggggttcgagtgggtctcaggctataatggtggtagtgatagggcaggctatacagactccgtgaagggccgattcaccatctccagagacaacgccaagaacacgctgtatctgcaaatgaacagcctgaaacctgaggacacggccgtgtattactgtgcaagatatatagcaaatagcggtacttactactgggactactggggccaggggaccctggtcaccgtgtcgagtgctagcaccaagggcccatcggtcttccccctggcaccctcctccaagagcacctctgggggcacagcggccctgggctgcctggtcaaggactacttccccgaacccgtgaccgtgtcgtggaactcaggcgccctgaccagcggcgtgcacaccttcccagctgtccttcagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 95 - DNA, 1353 bp, synthetic construct
  source 1..1353: mol_type = other DNA
gaggtgcagctggtgcagtctgggggaggcttggtgcagcctggggggtctctgagactctcctgtgcagtctctggattcaccttcagtagcagtgtcatgagctgggtccgccaggctccaggaaaggggttcgagtgggtctcaggctataatggtggtagtgatagggcaggctttgcagactccgtggagggccgattcaccatctccagagacaacgccaagaacacgctgtatctgcaaatgaacagcctgaaacctgaggacacggccgtgtattactgtgcaagatatatagcaaatagcggtacttactactgggactactggggccaggggaccctggtcaccgtgtcgagtgctagcaccaagggcccatcggtcttccccctggcaccctcctccaagagcacctctgggggcacagcggccctgggctgcctggtcaaggactacttccccgaacccgtgaccgtgtcgtggaactcaggcgccctgaccagcggcgtgcacaccttcccagctgtccttcagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 96 - DNA, 1353 bp, synthetic construct
  source 1..1353: mol_type = other DNA
caggtgcagctggtgcagtctgggggaggcttggtgcagcctggggggtctctgagactctcctgtgcagcctctggattcaccttcagtagcggtctcatgagctgggtccgccaggctccaggaaaggggttcgagtgggtctcaggctataatggtggtagtgatagggcaggctttgcagactccgtggagggccgattcaccatctccagagacaacgccaagaacacgctgtatctgcaaatgaacagcctgaaacctgaggacacggccgtgtattactgtgcaagatatatagcaaatagcggtacttactactgggactactggggccaggggaccctggtcaccgtgtcgagtgctagcaccaagggcccatcggtcttccccctggcaccctcctccaagagcacctctgggggcacagcggccctgggctgcctggtcaaggactacttccccgaacccgtgaccgtgtcgtggaactcaggcgccctgaccagcggcgtgcacaccttcccagctgtccttcagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 97 - DNA, 1353 bp, synthetic construct
  source 1..1353: mol_type = other DNA
gaagtgcagctggtgcagtctgggggagggttggtgcagtctggggggtctctgagagtctcctgtgcagcctctggattcaccttcagtagcggtctcatgagctgggtccgccaggctccaggaaaggggttcgagtgggtctcaggctataatggtggtagtgatagggcaggctacacagactccgtgaagggccgattcaccatctccagagacaacgccaagaacacgctgtatctgcaaatgaacagcctgaaacctgaggacacggccgtgtattactgtgcaagatatatagcgaatagcggtacttactactgggactactggggccaggggaccacggtcaccgtctcgagtgctagcaccaagggcccatcggtcttccccctggcaccctcctccaagagcacctctgggggcacagcggccctgggctgcctggtcaaggactacttccccgaacccgtgaccgtgtcgtggaactcaggcgccctgaccagcggcgtgcacaccttcccagctgtccttcagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 98 - DNA, 1353 bp, synthetic construct
  source 1..1353: mol_type = other DNA
gaagtgcagctggtgcagtctgggggagggttggtgcagtctggggggtctctgagagtctcctgtgcagcctctggattcaccttcagtagcggtctcatgagctgggtccgccaggctccaggaaaggggttcgagtgggtctcatgctataatggtggtagtgatagggcaggctacacagactccgtgaagggccgattcaccatctccagagacaacgccaagaacacgctgtatctgcaaatgaacagcctgaaacctgaggacacggccgtgtattactgtgcaagatatatagcgaatagcggtacttactactgggactactggggccaggggaccacggtcaccgtctcgagtgctagcaccaagggcccatcggtcttccccctggcaccctcctccaagagcacctctgggggcacagcggccctgggctgcctggtcaaggactacttccccgaacccgtgaccgtgtcgtggaactcaggcgccctgaccagcggcgtgcacaccttcccagctgtccttcagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 99 - DNA, 1353 bp, synthetic construct
  source 1..1353: mol_type = other DNA
gaggtgcagctggtgcagtctgggggaggcttggtgcagcctggggggtctctgagagtctcctgtgcagcctctggattcagcttcagtagcggtctcatgagctgggtccgccaggctccaggaaaggggttcgagtgggtctcaggctataatggtggtagtgatagggcaggctttgcagactccgtggagggccgattcaccatctccagagacaacgccaagaacacgctgtatctgcaaatgaacagcctgaaacctgaggacacggccgtgtattactgtgcaagatatatagcaaatagcggtacttactactgggactactggggccaggggaccctggtcaccgtctcgagtgctagcaccaagggcccatcggtcttccccctggcaccctcctccaagagcacctctgggggcacagcggccctgggctgcctggtcaaggactacttccccgaacccgtgaccgtgtcgtggaactcaggcgccctgaccagcggcgtgcacaccttcccagctgtccttcagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 100 - DNA, 1353 bp, synthetic construct
  source 1..1353: mol_type = other DNA
gaggtgcagctggtgcagtctgggggaggcttggtgcagcctggggggtctctgagactctcctgtgcagcctctggattcaccttcagtagcggtctcatgagctgggtccgccaggctccaggaaaggggttcgagtgggtctcaggctataatggtggtagtgatagggcaggctttgcagacgccgtggagggccgattcaccatctccagagacaacgccaagaacacgctgtatctgcaaatgaacagcctgaaacctgaggacacggccgtgtattactgtgcaagatatatagcaaatagcggtacttactactgggactactggggccaggggaccacggtcaccgtctcgagtgctagcaccaagggcccatcggtcttccccctggcaccctcctccaagagcacctctgggggcacagcggccctgggctgcctggtcaaggactacttccccgaacccgtgaccgtgtcgtggaactcaggcgccctgaccagcggcgtgcacaccttcccagctgtccttcagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgcagggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 101 - DNA, 651 bp, synthetic construct
  source 1..651: mol_type = other DNA
cagcctgtgctgactcagccaccctcagcgtctgggacccccgggcagagggtcaccatctcttgttctggaagcagctccaatatcgggagtaatactgtcaattggtatcagcaactcccaggaacggcccccaaactcctcatctataatgatagtcagcggccctcaggggtccctgaccaattctctgggtccaagtctggcacctcagcctccctggccatcagtgggctccagtctgacaatgaggctgattattactgtgcagcatgggatggcagcctgaatgtctggatgttcggcggagggaccaaggtcaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 102 - DNA, 651 bp, synthetic construct
  source 1..651: mol_type = other DNA
caggctgtgctgactcagcccccctcagtgtctgggacccccggacagagggtcaccatctcttgttctggaagcacctccaacatcggacataggactgtaaactggtatcagcaatttccaggagcggcccccaaactcctcatctataggaataatcagcggccctcaggggtccctgcccgattctctggctccaagtctggcacctcagcctccctggccatcagtgggctccagtctgaggatgaggctgattatcactgtgcagcatgggatgacagcctgactgcttgggtgttcggcggagggacccagctcaccgttttacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 103 - DNA, 651 bp, synthetic construct
  source 1..651: mol_type = other DNA
cagcctgtgctgactcagccaccctcggtgtctgaagcccccaggcagagggtcaccatctcctgttctggaagcagctccaacatcggaaataatgctgtaaactggtaccagcagctcccaggaaaggctcccaaactcctcatctttaagaataatcagcggccctcaggggtccctgaccggttctctggctccaagtctggcacctcagcctccctggtcatcagtgggctccagtctgaggatgaggctgattactactgtgcagcatgggatggcagcctgaatgcttggatgttcggcggggggaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 104 - DNA, 651 bp, synthetic construct
  source 1..651: mol_type = other DNA
cagtctgtgctgactcagccaccctcagcgtctgggacccccgggcagagggtcaccatctcttgttctggaagcagctccaacatcggaagtaatattgtaaactggtaccagcagttcccaggaacggcccccaaactcctcatttttagtaataatcagcggccctcaggggtccctgaccgattatctggctccaagtctggcacctcagcctccctggccatcagtgggctccagtctgaggatgaggctgattattactgttcagcatgggatcacagcctgaatgcctgggtgttcggcggagggaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 105 - DNA, 651 bp, synthetic construct
  source 1..651: mol_type = other DNA
caggctgtgctgactcagccaccctcagcgtctgggacccccgggcagagggtcagcatctcttgttctggaggcatctccaacatcggaagtaatattgtaaactggtaccaacacctcccaggaacggcccccaaactcctcatatatagtggtaatcaacggccctcaggagtccctgaccgattctctggctccaagtctggcacctcagcctccctggccatcagtgggctccagtctgaggatgaggctgattattactgtgcatcgtgggatgacagcctgaatggctgggtattcggcggagggaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 106 - DNA, 651 bp, synthetic construct
  source 1..651: mol_type = other DNA
caggctgtgctgactcagccaccctcagcgtctgggacccccgggcagagggtcagcatctcttgttctggaggcatctccaacatcggaagtaatattgtaaactggtaccaacacctcccaggaacggcccccaaactcctcatatatagtggtaatcaacggccctcaggagtccctgaccgattctctggctccaagtctggcacctcagcctccctggccatcagtggactccagtccgaggatgaggctgactattactgctcatcatgggacagcgacctcaggaattgggtgttcggcggggggaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 107 - DNA, 651 bp, synthetic construct
  source 1..651: mol_type = other DNA
cagcctgtgctgactcagccaccctcggtgtctgaagcccccaggcagagggtcaccatctcctgttctggaagcagctccaacatcggaaataatgctgtaaactggtaccagcagctcccaggaaaggctcccaaactcctcatctatagtactaatcagcggccctcaggggtccctgaccgattctctggttccaagtctggcacctcagcctccctggccatcagtgggctccagtctgaggatgaggctgattattactgtgcagcatgggatgacagagtgagtgcttggctgtttggcggagggaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 108 - DNA, 654 bp, synthetic construct
  source 1..654: mol_type = other DNA
cagtctgtgttgacgcagccgccctcagtgtctggggccccagggcagagggtcaccatctcctgcactgggagcagctccaacatcggggcaggttatgatgtacactggtaccagcaacttccaggaacagcccccaaactcgtcatctatggtaacaccaatcggccctcaggggtccctgaccgattctctggctccaagtctggcacctcagcctccctggccatcagtgggctccagtctgaggatgaggctgattattactgtgcagcatgggatgacagcctgaatggttgggtattcggcggaggcaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 109 - DNA, 651 bp, synthetic construct
  source 1..651: mol_type = other DNA
cagtctgccctgactcagccaccctcagcgtctgggacccccgggcagagggtcaccatctcttgttctggaagcatctccaacatcggaagtaatactgtaaactggtaccagcatctcccaggaacggcccccaaactcctcatctataatcataatcagcggccctcaggggtccctgaccgattctctggctccacgtctggcacgtcagcctccctggccatcagtgggctccagtctgaggatgaggctgattatttctgtgcaacatgggatgacagcctgagtgcttggctgttcggcggagggaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 110 - DNA, 651 bp, synthetic construct
  source 1..651: mol_type = other DNA
cagtctgtgctgacgcagccgccctcagcgtctgggacccccgggcagagggtcaccatctcttgttctggaagcagctccaacatcggaagtaatactgtgaactggtaccagcagctcccaggaacggcccccaaactcctcatctatagtgataatcagcggccctcaggtgtccctgaacgcttctctggctccaagtctggcacctcagcctccctggccatcagtggcctcctgtctgaggatgaggctgattattactgtgcagcatgggatggcagcctgaatgcttggctattcggcggtgggaccaaggtcaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 111 - DNA, 651 bp, synthetic construct
  source 1..651: mol_type = other DNA
tcctatgtgctgacacagccaccctcagcgtctgggacccccggacagagggtcaccatctcttgttctgggaccaggtccaacatcggaactaatactgttaactggtaccagcagctcccaggaacggcccccaaactcctcatctatagaaataatcagcggccctcaggggtccctgaccgattctctggctccaagtctggcacctcagcctccctggccatcagtgggctccagtctgaggatgaggctgattactactgtgcagtatgggatgacagagtgagtgcttggctgtttggcggagggaccaagctgaccgtcctacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

(19) United States
(12) Patent Application Publication
(10) Pub. No.: US 2025/0304707 A1
(43) Pub. Date: Oct. 2, 2025

(54) MONOCLONAL ANTIBODY OR ANTIGEN-BINDING FRAGMENT THEREOF THAT SPECIFICALLY BINDS TO BCMA, AND USE THEREOF

(71) Applicant: JOINT STOCK COMPANY "BIOCAD", pomeshch. 89 (RU)

(72) Inventors: Alina Valerevna BELIASNIKOVA, Saint Petersburg (RU); Olga Leonidovna KYTMANOVA, Saint Petersburg (RU); Anastasiya Andreevna IVANOVA, Saint Petersburg (RU); Valentina Yurevna FILINA, Vyborgskiy r-n (RU); Alina Sergeevna SAVINOVA, G. Krasnodar (RU); Dmitry Valentinovich MOROZOV, Saint Petersburg (RU)

(21) Appl. No.: 18/865,950

(22) PCT Filed: Jun. 11, 2023
(86) PCT No.: PCT/RU2023/050147

§ 371 Date: Nov. 14, 2024

(30) Foreign Application Priority Data
Jun. 9, 2022 (RU) 2022115671

(51) Int. Cl. C07K 16/28; A61K 45/06; C12N 15/70; C12N 15/85

(52) U.S. Cl. CPC C07K 16/2878; A61K 45/06; C12N 15/70; C12N 15/85; C07K 2317/14; C07K 2317/565; C07K 2317/76; C07K 2317/92

(57) ABSTRACT

The present invention relates to the field of biotechnology and medicine, in particular to a monoclonal antibody or antigen-binding fragment thereof that specifically binds to BCMA. The invention further relates to nucleic acids encoding said antibody, expression vectors, host cells and methods for producing same, methods for producing the antibodies according to the invention, pharmaceutical compositions comprising the antibody according to the invention, pharmaceutical compositions comprising the antibody according to the invention and other therapeutically active compounds, methods for treating BCMA-mediated diseases or disorders, uses of the antibodies or pharmaceutical compositions thereof for treating BCMA-mediated diseases or disorders, and uses of the antibodies and other therapeutically active compounds for treating BCMA-mediated diseases or disorders.

### FIELD OF THE INVENTION

The present invention relates to the field of biotechnology and medicine, in particular to a monoclonal antibody or antigen-binding fragment thereof that specifically binds to BCMA. The invention further relates to nucleic acids encoding said antibody, expression vectors, host cells and methods for producing same, methods for producing the antibodies according to the invention, pharmaceutical compositions comprising the antibody according to the invention, pharmaceutical compositions comprising the antibody according to the invention and other therapeutically active compounds, methods for treating BCMA-mediated diseases or disorders, uses of the antibodies or pharmaceutical compositions thereof for treating BCMA-mediated diseases or disorders, and uses of the antibodies and other therapeutically active compounds for treating BCMA-mediated diseases or disorders.

### BACKGROUND OF THE INVENTION

The B cell maturation antigen (BCMA, TNFRSF17 and CD269) is member of the tumor necrosis factor (TNF) receptor superfamily. Its native ligands are the B cell activating factor (BAFF; also called BLyS or TALL-1, TNFSF13B) and a proliferation-inducing ligand (APRIL, TNFSF13, CD256) which are involved in regulating various aspects of humoral immunity, B cell development, and homeostasis. However, APRIL binds to BCMA with significantly higher affinity (10⁻⁹ M) than BAFF (10⁻⁷ M) (Yu-Tzu Tai ET ALL, APRIL and BCMA promote human multiple myeloma growth and immunosuppression in the bone marrow microenvironment, Blood, 2016, 127, 25, pp. 3225-3236, https://doi.org/10.1182/blood-2016-01-691162).

BCMA is expressed at the late stages of B cell differentiation: on plasmablasts and plasmacytes (plasma cells, PCs). In multiple myeloma (MM), expression of BCMA is significantly increased on malignant plasmacytes versus normal cells, and activation of BCMA supports growth and survival of plasma cells via activating MEK/ERK, AKT, NFκB, JNK, p38, and Elk-1 (Shih-Feng Cho ET ALL, Targeting B Cell Maturation Antigen (BCMA) in Multiple Myeloma: Potential Uses of BCMA-Based Immunotherapy, Front Immunol, 2018, 9, 1821, https://www.ncbi.nlm.nih.gov/pmc/articles/PMC6095983).

BCMA is a promising target for immunotherapeutic products in various oncological diseases, for example, leukemia, lymphoma or multiple myeloma, as the antigen is characterized in limited abundance and increased expression on malignant plasma cells, as compared to normal plasmacytes.

Patent documents WO2012163805, WO2013072406, WO2014089335, WO2017143069 describe various antibodies to BCMA.

To date, in the world, only one anti-BCMA antibody as part of an antibody conjugated with a medicinal product has been approved for therapeutic use (Belantamab mafodotin). In connection with the above, there is a need for novel antibodies that specifically bind to BCMA.

### DISCLOSURE OF THE ESSENCE OF THE INVENTION

The authors of the present group of inventions have developed antibodies that specifically bind to BCMA and have high affinity parameters for binding to BCMA.

### Definitions and General Methods

Unless defined otherwise herein, all technical and scientific terms used in connection with the present invention will have the same meaning as is commonly understood by those skilled in the art.

Furthermore, unless otherwise required by context, singular terms shall include plural terms, and the plural terms shall include the singular terms. Typically, the present classification and methods of cell culture, molecular biology, immunology, microbiology, genetics, analytical chemistry, organic synthesis chemistry, medical and pharmaceutical chemistry, as well as hybridization and chemistry of protein and nucleic acids described herein are well known by those skilled and widely used in the art. Enzyme reactions and purification methods are performed according to the manufacturer's guidelines, as is common in the art, or as described herein.

The term “KD” in this description refers to the affinity constant (or equilibrium constant), which is calculated from the ratio of Kd to Ka (i.e. Kd/Ka), and it is expressed as a molar concentration (M).

“Binding affinity” generally refers to the strength of the sum total of noncovalent interactions between a single binding site of a molecule (e.g. an antibody) and its binding partner (e.g. an antigen). Unless indicated otherwise, “binding affinity” refers to intrinsic (characteristic, true) binding affinity which reflects a 1:1 interaction between members of a binding pair (e.g. antibody and antigen). The affinity of a molecule X for its binding partner Y can generally be represented by the affinity constant (KD). The preferred Kd value is about 200 nM, 150 nM, 100 nM, 60 nM, 50 nM, 40 nM, 30 nM, 20 nM, 10 nM, 8 nM, 6 nM, 4 nM, 2 nM, 1 nM, or less. Affinity can be measured by common methods known in the art, including those described in the present description. Low-affinity antibodies generally bind an antigen slowly and tend to dissociate readily, whereas high-affinity antibodies generally bind an antigen faster and tend to remain bound longer. A variety of methods for measuring binding affinity are known in the art, any one of these methods may be used for the purposes of the present invention.

The term “Kd”, “koff” or “kdis” refers to the off rate constant of a particular interaction between a binding molecule and antigen. The off rate constant koff can be measured using bio-layer interferometry, for example, using Octet™ system.

The term “Ka”, “kon” or “on-rate” refers to the association rate constant.

The term “R²” refers to the coefficient of determination.

The term “Response” refers to an antibody-antigen binding signal.

The term “in vitro” refers to a biological entity, a biological process, or a biological reaction outside the body under artificial conditions. For example, a cell grown in vitro is to be understood as a cell grown in an environment outside the body, e.g., in a test tube, a culture vial, or a microtiter plate.

The term “ED₅₀” (EC₅₀) (50% effective dose/concentration) refers to concentrations of a formulation producing 50% biological effect (which may include cytoxicity).

As used in the present description and claims that follow, unless otherwise dictated by the context, the words “include” and “comprise”, or variations thereof such as “includes”, “including”, “comprises”, or “comprising”, will be understood to imply the inclusion of a stated integer or group of integers but not the exclusion of any other integer or group of integers.

### Antibody

The present invention relates to a monoclonal antibody or antigen-binding fragment thereof that specifically binds to BCMA (B-cell maturation antigen; also known as TNFRSF17 and CD269).

The term “monoclonal antibody” or “mAb” refers to an antibody that is synthesized and isolated as an individual clonal population of cells.

The antibody of the invention is a recombinant antibody.

The term “recombinant antibody” refers to an antibody that is expressed in a cell or cell line comprising nucleotide sequence(s) encoding antibodies, wherein said nucleotide sequence(s) is (are) not associated with the cell in nature.

In one aspect, the present invention relates to an isolated monoclonal antibody or antigen-binding fragment thereof that specifically binds to BCMA, comprising:
(a) a heavy chain variable domain comprising:
(i) CDR1 with the amino acid sequence SX₁X₂MS, where
X₁=S or G;
X₂=A, L or V;
(ii) CDR2 with the amino acid sequence X₃YNGGSDRAGX₄X₅DSVX₆G, where
X₃=G or C;
X₄=F or Y;
X₅=A or T;
X₆=E or K; and
(iii) CDR3 with the amino acid sequence of SEQ ID NO: 7;
and
(b) a light chain variable domain comprising:
(i) CDR1 with the amino acid sequence X₇GX₈X₉SNIGX₁₀X₁₁X₁₂X₁₃VX₁₄, where
X₇=S or T;
X₈=S, G or T;
X₉=S, T, I or R;
X₁₀=0 or A;
X₁₁=S, H, N, G or T;
X₁₂=N, R or Y;
X₁₃=T, A, I or D;
X₁₄=N or H;
(ii) CDR2 with the amino acid sequence X₁₅X₁₆X₁₇X₁₈RPS, where
X₁₅=N, R, K, S or G;
X₁₆=D, N, G, T or H;
X₁₇=S, N or T;
X₁₈=Q or N;
and
(iii) CDR3 with the amino acid sequence X₁₉X₂₀WDX₂₁X₂₂X₂₃X₂₄X₂₅WX₂₆, where
X₁₉=A or S;
X₂₀=A, S, T or V;
X₂₁=G, D, H or S;
X₂₂=S, D or R;
X₂₃=L or V;
X₂₄=N, T, R or S;
X₂₅=V, A, G or N;
X₂₆=M, V or L.

The term “isolated” used to describe various antibodies according to the present description refers to an antibody which has been identified and isolated and/or regenerated from a cell or cell culture, in which the antibody is expressed. Impurities (contaminant components) from natural environment are materials which typically interfere with diagnostic or therapeutic uses of the polypeptide, and may include enzymes, hormones, and other proteinaceous or nonproteinaceous solutes. The isolated polypeptide is typically prepared by at least one purification step.

The term “antibody” or “immunoglobulin” (Ig) as used in the present description includes whole antibodies. The term “antibody” refers to a glycoprotein comprising at least two heavy (H) chains and two light (L) chains interconnected by disulfide bonds. Each heavy chain comprises a heavy chain variable region (abbreviated referred to in the present description as VH) and a heavy chain constant region. Each light chain consists of a light chain variable region (abbreviated referred to in the present description as VL) and light chain constant region. Preferably the light chain is a kappa (κ) light chain, and the constant domain CL is preferably C kappa (κ).

Antibodies according to the invention can be of any class (e.g., IgA, IgD, IgE, IgG, and IgM, preferably IgG), or subclass (e.g., IgG1, IgG2, IgG3, IgG4, IgA1 and IgA2, preferably IgG1).

VL and VH regions can be further subdivided into hyper-variability regions called complementarity determining regions (CDRs), interspersed between regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of heavy and light chains contain a binding domain that interacts with an antigen.

The constant regions of antibodies may mediate the binding of immunoglobulin to host tissues or factors, including various cells of the immune system (e.g. effector cells) and the first component (C1q) of the classical complement system.

The term “antigen-binding portion” of antibody or “antigen-binding fragment”, as used in the present description, refers to one or more antibody fragments that retain the ability to specifically bind to an antigen. It has been shown that the antigen-binding function of antibody can be performed by fragments of a full-length antibody. Examples of binding fragments which are included within the term “antigen-binding portion” of an antibody include (i) Fab-fragment, monovalent fragment, consisting of VL, VH, CL and CH1 domains; (ii) F(ab′)2 fragment, a bivalent fragment comprising two Fab-fragments linked by a disulfide bridge at the hinge region; (iii) Fd-fragment consisting of VH and CH1 domains; (iv) Fv-fragment consisting of VL and VH domains of a single arm of an antibody; (v) dAb-fragment (Ward et al., (1989) Nature 341:544-546), which consists of a VH/VHH domain. In addition, two regions of the Fv-fragment, VL and VH, are encoded by different genes, they can be joined using recombinant methods using a synthetic linker that enables to receive them as a single protein chain in which the VL and VH regions are paired to form monovalent molecules (known as a single-chain Fv (scFv); see e.g. Bird et al. (1988) Science 242:423-426; and Huston et al. (1988) Proc. Natl. Acad. Sci. USA 85:5879-5883). It is assumed that such single-stranded molecules are also included within the term “antigen-binding portion” of antibody. Such antibody fragments are produced using conventional techniques known to those skilled in the art, and these fragments are screened in the same manner as intact antibodies are.

“Kabat numbering scheme” or “numbering according to Kabat” as used in the present application refers to the system for numbering of amino acid residues that are more variable (i.e. hypervariable) than other amino acid residues in variable regions of heavy and light chains of antibody (Kabat et al. Ann. N.Y. Acad. Sci., 190:382-93 (1971); Kabat et al. Sequences of Proteins of Immunological Interest, Fifth Edition, U.S. Department of Health and Human Services, NIH Publication No. 91-3242 (1991)).

The antibody of the present invention “which specifically binds” a target antigen refers to an antibody that binds an antigen with sufficient affinity such that the antibody can be used as a diagnostic and/or therapeutic agent targeting a protein or cell or tissue expressing the antigen, and slightly cross-reacts with other proteins.

The term “specifically binds to” a particular polypeptide or an epitope on a particular target polypeptide may be described by example of a molecule having a Kd for the target of at least about 200 nM, or at least about 150 nM, or at least about 100 nM, or at least about 60 nM, or at least about 50 nM, or at least about 40 nM, or at least about 30 nM, or at least about 20 nM, or at least about 10 nM, or at least about 8 nM, or at least about 6 nM, or at least about 4 nM, or at least about 2 nM, or at least about 1 nM, or greater.

In one embodiment, the term “specific binding” refers to binding where a molecule binds to a particular polypeptide or epitope on a particular polypeptide without substantially binding to any other polypeptide or epitope on a polypeptide.

In some embodiments of the invention, the isolated monoclonal antibody or antigen-binding fragment thereof includes a heavy chain variable domain that comprises CDR1 with an amino acid sequence that is selected from the group: SEQ ID NO: 1, SEQ ID NO: 2 or SEQ ID NO: 3.

In some embodiments of the invention, the isolated monoclonal antibody or antigen-binding fragment thereof includes a heavy chain variable domain that comprises CDR2 with an amino acid sequence that is selected from the group: SEQ ID NO: 4, SEQ ID NO: 5 or SEQ ID NO: 6.

In some embodiments of the invention, the isolated monoclonal antibody or antigen-binding fragment thereof includes a light chain variable domain that comprises CDR1 with an amino acid sequence that is selected from the group: SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15.

In some embodiments of the invention, the isolated monoclonal antibody or antigen-binding fragment thereof includes a light chain variable domain that comprises CDR2 with an amino acid sequence that is selected from the group: SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 or SEQ ID NO: 24.

In some embodiments of the invention, the isolated monoclonal antibody or antigen-binding fragment thereof includes a light chain variable domain that comprises CDR3 with an amino acid sequence that is selected from the group: SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 or SEQ ID NO: 35.

In some embodiments of the invention, the isolated monoclonal antibody or antigen-binding fragment thereof includes a heavy chain variable domain comprising:
CDR1 with an amino acid sequence that is selected from the group: SEQ ID NO: 1, SEQ ID NO: 2 or SEQ ID NO: 3;
CDR2 with an amino acid sequence that is selected from the group: SEQ ID NO: 4, SEQ ID NO: 5 or SEQ ID NO: 6;
and
CDR3 with the amino acid sequence of SEQ ID NO: 7.

In some embodiments of the invention, the isolated monoclonal antibody or antigen-binding fragment thereof includes:
(i) a heavy chain variable domain comprising:
CDR1 with the amino acid sequence of SEQ ID NO: 1,
CDR2 with the amino acid sequence of SEQ ID NO: 4 and
CDR3 with the amino acid sequence of SEQ ID NO: 7;
(ii) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7;
(iii) a heavy chain variable domain comprising:
CDR1 with the amino acid sequence of SEQ ID NO: 3,
CDR2 with the amino acid sequence of SEQ ID NO: 4 and
CDR3 with the amino acid sequence of SEQ ID NO: 7;
(iv) a heavy chain variable domain comprising:
CDR1 with the amino acid sequence of SEQ ID NO: 2,
CDR2 with the amino acid sequence of SEQ ID NO: 4 and
CDR3 with the amino acid sequence of SEQ ID NO: 7;
or
(v) a heavy chain variable domain comprising:
CDR1 with the amino acid sequence of SEQ ID NO: 2,
CDR2 with the amino acid sequence of SEQ ID NO: 6 and
CDR3 with the amino acid sequence of SEQ ID NO: 7.

In some embodiments of the invention, the isolated monoclonal antibody or antigen-binding fragment thereof includes a light chain variable domain comprising:
CDR1 with an amino acid sequence that is selected from the group: SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15;
CDR2 with an amino acid sequence that is selected from the group: SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 or SEQ ID NO: 24;
and
CDR3 with an amino acid sequence that is selected from the group: SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 or SEQ ID NO: 35.

In some embodiments of the invention, the isolated monoclonal antibody or antigen-binding fragment thereof includes:
(i) a light chain variable domain comprising:
CDR1 with the amino acid sequence of SEQ ID NO: 8,
CDR2 with the amino acid sequence of SEQ ID NO: 16 and
CDR3 with the amino acid sequence of SEQ ID NO: 25;
(ii) a light chain variable domain comprising:
CDR1 with the amino acid sequence of SEQ ID NO: 9,
CDR2 with the amino acid sequence of SEQ ID NO: 17 and
CDR3 with the amino acid sequence of SEQ ID NO: 26;
(iii) a light chain variable domain comprising:
CDR1 with the amino acid sequence of SEQ ID NO: 10,
CDR2 with the amino acid sequence of SEQ ID NO: 18 and
CDR3 with the amino acid sequence of SEQ ID NO: 27;
(iv) a light chain variable domain comprising:
CDR1 with the amino acid sequence of SEQ ID NO: 11,
CDR2 with the amino acid sequence of SEQ ID NO: 19 and
CDR3 with the amino acid sequence of SEQ ID NO: 28;
(v) a light chain variable domain comprising:
CDR1 with the amino acid sequence of SEQ ID NO: 12,
CDR2 with the amino acid sequence of SEQ ID NO: 20 and
CDR3 with the amino acid sequence of SEQ ID NO: 29;
(vi) a light chain variable domain comprising:
CDR1 with the amino acid sequence of SEQ ID NO: 12,
CDR2 with the amino acid sequence of SEQ ID NO: 20 and
CDR3 with the amino acid sequence of SEQ ID NO: 30;
(vii) a light chain variable domain comprising:
CDR1 with the amino acid sequence of SEQ ID NO: 10,
CDR2 with the amino acid sequence of SEQ ID NO: 21 and
CDR3 with the amino acid sequence of SEQ ID NO: 31;
(viii) a light chain variable domain comprising:
CDR1 with the amino acid sequence of SEQ ID NO: 13,
CDR2 with the amino acid sequence of SEQ ID NO: 22 and
CDR3 with the amino acid sequence of SEQ ID NO: 32;
(ix) a light chain variable domain comprising:
CDR1 with the amino acid sequence of SEQ ID NO: 14,
CDR2 with the amino acid sequence of SEQ ID NO: 23 and
CDR3 with the amino acid sequence of SEQ ID NO: 33;
(x) a light chain variable domain comprising:
CDR1 with the amino acid sequence of SEQ ID NO: 8,
CDR2 with the amino acid sequence of SEQ ID NO: 24 and
CDR3 with the amino acid sequence of SEQ ID NO: 34;
or
(xi) a light chain variable domain comprising:
CDR1 with the amino acid sequence of SEQ ID NO: 15,
CDR2 with the amino acid sequence of SEQ ID NO: 17 and
CDR3 with the amino acid sequence of SEQ ID NO: 35.

In some embodiments of the invention, the isolated monoclonal antibody or antigen-binding fragment thereof includes:
(a) a heavy chain variable domain comprising:
CDR1 with an amino acid sequence that is selected from the group: SEQ ID NO: 1, SEQ ID NO: 2 or SEQ ID NO: 3;
CDR2 with an amino acid sequence that is selected from the group: SEQ ID NO: 4, SEQ ID NO: 5 or SEQ ID NO: 6;
and
CDR3 with the amino acid sequence of SEQ ID NO: 7; and
(b) a light chain variable domain comprising:
CDR1 with an amino acid sequence that is selected from the group: SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15;
CDR2 with an amino acid sequence that is selected from the group: SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 or SEQ ID NO: 24;
and
CDR3 with an amino acid sequence that is selected from the group: SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 or SEQ ID NO: 35.

In some embodiments of the invention, the isolated monoclonal antibody or antigen-binding fragment thereof includes:
(i) (a) a heavy chain variable domain comprising:
CDR1 with the amino acid sequence of SEQ ID NO: 1,
CDR2 with the amino acid sequence of SEQ ID NO: 4 and
CDR3 with the amino acid sequence of SEQ ID NO: 7; and
(b) a light chain variable domain comprising:
CDR1 with the amino acid sequence of SEQ ID NO: 8,
CDR2 with the amino acid sequence of SEQ ID NO: 16 and
CDR3 with the amino acid sequence of SEQ ID NO: 25;
(ii) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7; and
(b) a light chain variable domain comprising:
CDR1 with the amino acid sequence of SEQ ID NO: 9,
CDR2 with the amino acid sequence of SEQ ID NO: 17 and
CDR3 with the amino acid sequence of SEQ ID NO: 26;
(iii) (a) a heavy chain variable domain comprising:
CDR1 with the amino acid sequence of SEQ ID NO: 3,
CDR2 with the amino acid sequence of SEQ ID NO: 4 and
CDR3 with the amino acid sequence of SEQ ID NO: 7; and
(b) a light chain variable domain comprising:
CDR1 with the amino acid sequence of SEQ ID NO: 10,
CDR2 with the amino acid sequence of SEQ ID NO: 18 and
CDR3 with the amino acid sequence of SEQ ID NO: 27;
(iv) (a) a heavy chain variable domain comprising:
CDR1 with the amino acid sequence of SEQ ID NO: 2,
CDR2 with the amino acid sequence of SEQ ID NO: 4 and
CDR3 with the amino acid sequence of SEQ ID NO: 7; and
(b) a light chain variable domain comprising:
CDR1 with the amino acid sequence of SEQ ID NO: 11,
CDR2 with the amino acid sequence of SEQ ID NO: 19 and
CDR3 with the amino acid sequence of SEQ ID NO: 28;
(v) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7; and
(b) a light chain variable domain comprising:
CDR1 with the amino acid sequence of SEQ ID NO: 12,
CDR2 with the amino acid sequence of SEQ ID NO: 20 and
CDR3 with the amino acid sequence of SEQ ID NO: 29;
(vi) (a) a heavy chain variable domain comprising:
CDR1 with the amino acid sequence of SEQ ID NO: 2,
CDR2 with the amino acid sequence of SEQ ID NO: 6 and
CDR3 with the amino acid sequence of SEQ ID NO: 7; and
(b) a light chain variable domain comprising:
CDR1 with the amino acid sequence of SEQ ID NO: 12,
CDR2 with the amino acid sequence of SEQ ID NO: 20 and
CDR3 with the amino acid sequence of SEQ ID NO: 30;
(vii) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7; and
(b) a light chain variable domain comprising:
CDR1 with the amino acid sequence of SEQ ID NO: 10,
CDR2 with the amino acid sequence of SEQ ID NO: 21 and
CDR3 with the amino acid sequence of SEQ ID NO: 31;
(viii) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7, and
(b) a light chain variable domain comprising:
CDR1 with the amino acid sequence of SEQ ID NO: 13,
CDR2 with the amino acid sequence of SEQ ID NO: 22 and
CDR3 with the amino acid sequence of SEQ ID NO: 32;
(ix) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7; and
(b) a light chain variable domain comprising:
CDR1 with the amino acid sequence of SEQ ID NO: 12,
CDR2 with the amino acid sequence of SEQ ID NO: 20 and
CDR3 with the amino acid sequence of SEQ ID NO: 30;
(x) (a) a heavy chain variable domain comprising:
CDR1 with the amino acid sequence of SEQ ID NO: 2,
CDR2 with the amino acid sequence of SEQ ID NO: 4 and
CDR3 with the amino acid sequence of SEQ ID NO: 7; and
(b) a light chain variable domain comprising:
CDR1 with the amino acid sequence of SEQ ID NO: 14,
CDR2 with the amino acid sequence of SEQ ID NO: 23 and
CDR3 with the amino acid sequence of SEQ ID NO: 33;
(xi) (a) a heavy chain variable domain comprising:
CDR1 with the amino acid sequence of SEQ ID NO: 2,
CDR2 with the amino acid sequence of SEQ ID NO: 4 and
CDR3 with the amino acid sequence of SEQ ID NO: 7; and
(b) a light chain variable domain comprising:
CDR1 with the amino acid sequence of SEQ ID NO: 8,
CDR2 with the amino acid sequence of SEQ ID NO: 24 and
CDR3 with the amino acid sequence of SEQ ID NO: 34;
or
(xii) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7; and
(b) a light chain variable domain comprising:
CDR1 with the amino acid sequence of SEQ ID NO: 15,
CDR2 with the amino acid sequence of SEQ ID NO: 17 and
CDR3 with the amino acid sequence of SEQ ID NO: 35.

In some embodiments of the invention, the isolated monoclonal antibody or antigen-binding fragment thereof includes a heavy chain variable domain that comprises an amino acid sequence that is selected from the group: SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43.

In some embodiments of the invention, the isolated monoclonal antibody or antigen-binding fragment thereof includes a light chain variable domain that comprises an amino acid sequence that is selected from the group: SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53 or SEQ ID NO: 54.

In some embodiments of the invention, the isolated monoclonal antibody or antigen-binding fragment thereof includes:
(a) a heavy chain variable domain that comprises an amino acid sequence that is selected from the group: SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43; and
(b) a light chain variable domain that comprises an amino acid sequence that is selected from the group: SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53 or SEQ ID NO: 54.

In some embodiments of the invention, the isolated monoclonal antibody or antigen-binding fragment thereof includes:
(i) a heavy chain variable domain that comprises the amino acid sequence of SEQ ID NO: 36 and
(b) a light chain variable domain that comprises the amino acid sequence of SEQ ID NO: 44;
(ii) (a) a heavy chain variable domain that comprises the amino acid sequence of SEQ ID NO: 37 and
(b) a light chain variable domain that comprises the amino acid sequence of SEQ ID NO: 45;
(iii) (a) a heavy chain variable domain that comprises the amino acid sequence of SEQ ID NO: 38 and
(b) a light chain variable domain that comprises the amino acid sequence of SEQ ID NO: 46;
(iv) (a) a heavy chain variable domain that comprises the amino acid sequence of SEQ ID NO: 39 and
(b) a light chain variable domain that comprises the amino acid sequence of SEQ ID NO: 47;
(v) (a) a heavy chain variable domain that comprises the amino acid sequence of SEQ ID NO: 40 and
(b) a light chain variable domain that comprises the amino acid sequence of SEQ ID NO: 48;
(vi) (a) a heavy chain variable domain that comprises the amino acid sequence of SEQ ID NO: 41 and
(b) a light chain variable domain that comprises the amino acid sequence of SEQ ID NO: 49;
(vii) (a) a heavy chain variable domain that comprises the amino acid sequence of SEQ ID NO: 40 and
(b) a light chain variable domain that comprises the amino acid sequence of SEQ ID NO: 50;
(viii) (a) a heavy chain variable domain that comprises the amino acid sequence of SEQ ID NO: 40 and
(b) a light chain variable domain that comprises the amino acid sequence of SEQ ID NO: 51;
(ix) (a) a heavy chain variable domain that comprises the amino acid sequence of SEQ ID NO: 40 and
(b) a light chain variable domain that comprises the amino acid sequence of SEQ ID NO: 49;
(x) (a) a heavy chain variable domain that comprises the amino acid sequence of SEQ ID NO: 42 and
(b) a light chain variable domain that comprises the amino acid sequence of SEQ ID NO: 52;
(xi) (a) a heavy chain variable domain that comprises the amino acid sequence of SEQ ID NO: 43 and
(b) a light chain variable domain that comprises the amino acid sequence of SEQ ID NO: 53;
or
(xii) (a) a heavy chain variable domain that comprises the amino acid sequence of SEQ ID NO: 40 and
(b) a light chain variable domain that comprises the amino acid sequence of SEQ ID NO: 54.

In some embodiments of the invention, the isolated monoclonal antibody that specifically binds to BCMA is a full-length IgG antibody.

In some embodiments of the invention, the isolated monoclonal antibody that specifically binds to BCMA is a full-length IgG antibody that is of human IgG1, IgG2, IgG3 or IgG4 isotype.

In some embodiments of the invention, the isolated monoclonal antibody that specifically binds to BCMA is a full-length IgG antibody that is of human IgG1 isotype.

In some embodiments of the invention, the isolated monoclonal antibody comprises mutations L234A and L235A (or L247A and L248A according to Kabat) in CH2 according to EU numbering of antibody amino acids (Edelman G. M. et al., Proc. Natl. Acad. Sci. USA 63 (1969) pp. 78-85; Kabat, E. A., et al., Sequences of Proteins of Immunological Interest, 5th ed., Public Health Service, National Institutes of Health, Bethesda, MD, (1991).

In some embodiments of the invention, the isolated monoclonal antibody includes a heavy chain comprising an amino acid sequence that is selected from the group: SEQ ID NO: 55, SEQ ID NO: 56, SEQ ID NO: 57, SEQ ID NO: 58, SEQ ID NO: 59, SEQ ID NO: 60, SEQ ID NO: 61 or SEQ ID NO: 62.

In some embodiments of the invention, the isolated monoclonal antibody includes a light chain comprising an amino acid sequence that is selected from the group: SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72 or SEQ ID NO: 73.

In some embodiments of the invention, the isolated monoclonal antibody includes:
(i) (a) a heavy chain comprising an amino acid sequence that is selected from the group: SEQ ID NO: 55, SEQ ID NO: 56, SEQ ID NO: 57, SEQ ID NO: 58, SEQ ID NO: 59, SEQ ID NO: 60, SEQ ID NO: 61 or SEQ ID NO: 62, and
(b) a light chain comprising an amino acid sequence that is selected from the group: SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72 or SEQ ID NO: 73.

In some embodiments of the invention, the isolated monoclonal antibody includes:
(i) (a) a heavy chain comprising the amino acid sequence of SEQ ID NO: 55, and
(b) a light chain comprising the amino acid sequence of SEQ ID NO: 63;
(ii) (a) a heavy chain comprising the amino acid sequence of SEQ ID NO: 56, and
(b) a light chain comprising the amino acid sequence of SEQ ID NO: 64;
(iii) (a) a heavy chain comprising the amino acid sequence of SEQ ID NO: 57, and
(b) a light chain comprising the amino acid sequence of SEQ ID NO: 65;
(iv) (a) a heavy chain comprising the amino acid sequence of SEQ ID NO: 58, and
(b) a light chain comprising the amino acid sequence of SEQ ID NO: 66;
(v) (a) a heavy chain comprising the amino acid sequence of SEQ ID NO: 59, and
(b) a light chain comprising the amino acid sequence of SEQ ID NO: 67;
(vi) (a) a heavy chain comprising the amino acid sequence of SEQ ID NO: 60, and
(b) a light chain comprising the amino acid sequence of SEQ ID NO: 68;
(vii) (a) a heavy chain comprising the amino acid sequence of SEQ ID NO: 59, and
(b) a light chain comprising the amino acid sequence of SEQ ID NO: 69;
(viii) (a) a heavy chain comprising the amino acid sequence of SEQ ID NO: 59, and
(b) a light chain comprising the amino acid sequence of SEQ ID NO: 70;
(ix) (a) a heavy chain comprising the amino acid sequence of SEQ ID NO: 59, and
(b) a light chain comprising the amino acid sequence of SEQ ID NO: 68,
(x) (a) a heavy chain comprising the amino acid sequence of SEQ ID NO: 61, and
(b) a light chain comprising the amino acid sequence of SEQ ID NO: 71;
(xi) (a) a heavy chain comprising the amino acid sequence of SEQ ID NO: 62, and
(b) a light chain comprising the amino acid sequence of SEQ ID NO: 72;
or
(xii) (a) a heavy chain comprising the amino acid sequence of SEQ ID NO: 59, and
(b) a light chain comprising the amino acid sequence of SEQ ID NO: 73.

In some embodiments of the invention, the isolated monoclonal antibody that specifically binds to BCMA is antibody 01-001.

Antibody 01-001 includes:
(a) a heavy chain comprising the amino acid sequence of SEQ ID NO: 55; and
(b) a light chain comprising the amino acid sequence of SEQ ID NO: 63.

Antibody 01-001 includes:
(a) a heavy chain variable domain that comprises the amino acid sequence of SEQ ID NO: 36;
(b) a light chain variable domain that comprises the amino acid sequence of SEQ ID NO: 44.

Antibody 01-001 includes:
(a) a heavy chain variable domain comprising:
(i) CDR1 (Kabat) with the amino acid sequence of SEQ ID NO: 1,
(ii) CDR2 (Kabat) with the amino acid sequence of SEQ ID NO: 4,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 8,
(ii) CDR2 (Kabat) with the amino acid sequence of SEQ ID NO: 16,
(iii) CDR3 (Kabat) with the amino acid sequence of SEQ ID NO: 25.

In some embodiments of the invention, the isolated monoclonal antibody that specifically binds to BCMA is antibody 01-006.

Antibody 01-006 includes:
(a) a heavy chain comprising the amino acid sequence of SEQ ID NO: 56; and
(b) a light chain comprising the amino acid sequence of SEQ ID NO: 64.

Antibody 01-006 includes:
(a) a heavy chain variable domain that comprises the amino acid sequence of SEQ ID NO: 37;
(b) a light chain variable domain that comprises the amino acid sequence of SEQ ID NO: 45.

Antibody 01-006 includes:
(a) a heavy chain variable domain comprising:
(i) CDR1 (Kabat) with the amino acid sequence of SEQ ID NO: 2,
(ii) CDR2 (Kabat) with the amino acid sequence of SEQ ID NO: 5,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 9,
(ii) CDR2 (Kabat) with the amino acid sequence of SEQ ID NO: 17,
(iii) CDR3 (Kabat) with the amino acid sequence of SEQ ID NO: 26.

In some embodiments of the invention, the isolated monoclonal antibody that specifically binds to BCMA is antibody 01-016.

Antibody 01-016 includes:
(a) a heavy chain comprising the amino acid sequence of SEQ ID NO: 57; and
(b) a light chain comprising the amino acid sequence of SEQ ID NO: 65.

Antibody 01-016 includes:
(a) a heavy chain variable domain that comprises the amino acid sequence of SEQ ID NO: 38;
(b) a light chain variable domain that comprises the amino acid sequence of SEQ ID NO: 46.

Antibody 01-016 includes:
(a) a heavy chain variable domain comprising:
(i) CDR1 (Kabat) with the amino acid sequence of SEQ ID NO: 3,
(ii) CDR2 (Kabat) with the amino acid sequence of SEQ ID NO: 4,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 10,
(ii) CDR2 (Kabat) with the amino acid sequence of SEQ ID NO: 18,
(iii) CDR3 (Kabat) with the amino acid sequence of SEQ ID NO: 27.

In some embodiments of the invention, the isolated monoclonal antibody that specifically binds to BCMA is antibody 01-021.

Antibody 01-021 includes:
(a) a heavy chain comprising the amino acid sequence of SEQ ID NO: 58; and
(b) a light chain comprising the amino acid sequence of SEQ ID NO: 66.

Antibody 01-021 includes:
(a) a heavy chain variable domain that comprises the amino acid sequence of SEQ ID NO: 39;
(b) a light chain variable domain that comprises the amino acid sequence of SEQ ID NO: 47.

Antibody 01-021 includes:
(a) a heavy chain variable domain comprising:
(i) CDR1 (Kabat) with the amino acid sequence of SEQ ID NO: 2,
(ii) CDR2 (Kabat) with the amino acid sequence of SEQ ID NO: 4,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 11,
(ii) CDR2 (Kabat) with the amino acid sequence of SEQ ID NO: 19,
(iii) CDR3 (Kabat) with the amino acid sequence of SEQ ID NO: 28.

In some embodiments of the invention, the isolated monoclonal antibody that specifically binds to BCMA is antibody 01-022.

Antibody 01-022 includes:
(a) a heavy chain comprising the amino acid sequence of SEQ ID NO: 59; and
(b) a light chain comprising the amino acid sequence of SEQ ID NO: 67.

Antibody 01-022 includes:
(a) a heavy chain variable domain that comprises the amino acid sequence of SEQ ID NO: 40;
(b) a light chain variable domain that comprises the amino acid sequence of SEQ ID NO: 48.

Antibody 01-022 includes:
(a) a heavy chain variable domain comprising:
(i) CDR1 (Kabat) with the amino acid sequence of SEQ ID NO: 2,
(ii) CDR2 (Kabat) with the amino acid sequence of SEQ ID NO: 5,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 12,
(ii) CDR2 (Kabat) with the amino acid sequence of SEQ ID NO: 20,
(iii) CDR3 (Kabat) with the amino acid sequence of SEQ ID NO: 29.

In some embodiments of the invention, the isolated monoclonal antibody that specifically binds to BCMA is antibody 01-024.

Antibody 01-024 includes:
(a) a heavy chain comprising the amino acid sequence of SEQ ID NO: 60; and
(b) a light chain comprising the amino acid sequence of SEQ ID NO: 68.

Antibody 01-024 includes:
(a) a heavy chain variable domain that comprises the amino acid sequence of SEQ ID NO: 41;
(b) a light chain variable domain that comprises the amino acid sequence of SEQ ID NO: 49.

Antibody 01-024 includes:
(a) a heavy chain variable domain comprising:
(i) CDR1 (Kabat) with the amino acid sequence of SEQ ID NO: 2,
(ii) CDR2 (Kabat) with the amino acid sequence of SEQ ID NO: 6,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 12,
(ii) CDR2 (Kabat) with the amino acid sequence of SEQ ID NO: 20,
(iii) CDR3 (Kabat) with the amino acid sequence of SEQ ID NO: 30.

In some embodiments of the invention, the isolated monoclonal antibody that specifically binds to BCMA is antibody 01-025.

Antibody 01-025 includes:
(a) a heavy chain comprising the amino acid sequence of SEQ ID NO: 59; and
(b) a light chain comprising the amino acid sequence of SEQ ID NO: 69.

Antibody 01-025 includes:
(a) a heavy chain variable domain that comprises the amino acid sequence of SEQ ID NO: 40;
(b) a light chain variable domain that comprises the amino acid sequence of SEQ ID NO: 50.

Antibody 01-025 includes:
(a) a heavy chain variable domain comprising:
(i) CDR1 (Kabat) with the amino acid sequence of SEQ ID NO: 2,
(ii) CDR2 (Kabat) with the amino acid sequence of SEQ ID NO: 5,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 10,
(ii) CDR2 (Kabat) with the amino acid sequence of SEQ ID NO: 21,
(iii) CDR3 (Kabat) with the amino acid sequence of SEQ ID NO: 31.

In some embodiments of the invention, the isolated monoclonal antibody that specifically binds to BCMA is antibody 01-026.

Antibody 01-026 includes:
(a) a heavy chain comprising the amino acid sequence of SEQ ID NO: 59; and
(b) a light chain comprising the amino acid sequence of SEQ ID NO: 70.

Antibody 01-026 includes:
(a) a heavy chain variable domain that comprises the amino acid sequence of SEQ ID NO: 40;
(b) a light chain variable domain that comprises the amino acid sequence of SEQ ID NO: 51.

Antibody 01-026 includes:
(a) a heavy chain variable domain comprising:
(i) CDR1 (Kabat) with the amino acid sequence of SEQ ID NO: 2,
(ii) CDR2 (Kabat) with the amino acid sequence of SEQ ID NO: 5,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 13,
(ii) CDR2 (Kabat) with the amino acid sequence of SEQ ID NO: 22,
(iii) CDR3 (Kabat) with the amino acid sequence of SEQ ID NO: 32.

In some embodiments of the invention, the isolated monoclonal antibody that specifically binds to BCMA is antibody 01-027.

Antibody 01-027 includes:
(a) a heavy chain comprising the amino acid sequence of SEQ ID NO: 59; and
(b) a light chain comprising the amino acid sequence of SEQ ID NO: 68.

Antibody 01-027 includes:
(a) a heavy chain variable domain that comprises the amino acid sequence of SEQ ID NO: 40;
(b) a light chain variable domain that comprises the amino acid sequence of SEQ ID NO: 49.

Antibody 01-027 includes:
(a) a heavy chain variable domain comprising:
(i) CDR1 (Kabat) with the amino acid sequence of SEQ ID NO: 2,
(ii) CDR2 (Kabat) with the amino acid sequence of SEQ ID NO: 5,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 12,
(ii) CDR2 (Kabat) with the amino acid sequence of SEQ ID NO: 20,
(iii) CDR3 (Kabat) with the amino acid sequence of SEQ ID NO: 30.

In some embodiments of the invention, the isolated monoclonal antibody that specifically binds to BCMA is antibody 01-030.

Antibody 01-030 includes:
(a) a heavy chain comprising the amino acid sequence of SEQ ID NO: 61; and
(b) a light chain comprising the amino acid sequence of SEQ ID NO: 71.

Antibody 01-030 includes:
(a) a heavy chain variable domain that comprises the amino acid sequence of SEQ ID NO: 42;
(b) a light chain variable domain that comprises the amino acid sequence of SEQ ID NO: 52.

Antibody 01-030 includes:
(a) a heavy chain variable domain comprising:
(i) CDR1 (Kabat) with the amino acid sequence of SEQ ID NO: 2,
(ii) CDR2 (Kabat) with the amino acid sequence of SEQ ID NO: 4,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 14,
(ii) CDR2 (Kabat) with the amino acid sequence of SEQ ID NO: 23,
(iii) CDR3 (Kabat) with the amino acid sequence of SEQ ID NO: 33.

In some embodiments of the invention, the isolated monoclonal antibody that specifically binds to BCMA is antibody 01-033.

Antibody 01-033 includes:
(a) a heavy chain comprising the amino acid sequence of SEQ ID NO: 62; and
(b) a light chain comprising the amino acid sequence of SEQ ID NO: 72.

Antibody 01-033 includes:
(a) a heavy chain variable domain that comprises the amino acid sequence of SEQ ID NO: 43;
(b) a light chain variable domain that comprises the amino acid sequence of SEQ ID NO: 53.

Antibody 01-033 includes:
(a) a heavy chain variable domain comprising:
(i) CDR1 (Kabat) with the amino acid sequence of SEQ ID NO: 2,
(ii) CDR2 (Kabat) with the amino acid sequence of SEQ ID NO: 4,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 8,
(ii) CDR2 (Kabat) with the amino acid sequence of SEQ ID NO: 24,
(iii) CDR3 (Kabat) with the amino acid sequence of SEQ ID NO: 34.

In some embodiments of the invention, the isolated monoclonal antibody that specifically binds to BCMA is antibody 01-037.

Antibody 01-037 includes:
(a) a heavy chain comprising the amino acid sequence of SEQ ID NO: 59; and
(b) a light chain comprising the amino acid sequence of SEQ ID NO: 73.

Antibody 01-037 includes:
(a) a heavy chain variable domain that comprises the amino acid sequence of SEQ ID NO: 40;
(b) a light chain variable domain that comprises the amino acid sequence of SEQ ID NO: 54.

Antibody 01-037 includes:
(a) a heavy chain variable domain comprising:
(i) CDR1 (Kabat) with the amino acid sequence of SEQ ID NO: 2,
(ii) CDR2 (Kabat) with the amino acid sequence of SEQ ID NO: 5,
(iii) CDR3 (Kabat) with the amino acid sequence of SEQ ID NO: 7, and
(b) a light chain variable domain comprising:
(i) CDR1 (Kabat) with the amino acid sequence of SEQ ID NO: 15,
(ii) CDR2 (Kabat) with the amino acid sequence of SEQ ID NO: 17,
(iii) CDR3 (Kabat) with the amino acid sequence of SEQ ID NO: 35.

### Nucleic Acid Molecule

In one aspect, the present invention relates to a nucleic acid that encodes any one of the above antibody or antigen-binding fragment thereof that specifically binds to BCMA.

In any one of said embodiments, the nucleic acid molecules may be isolated.

The terms “nucleic acid”, “nucleic sequence”, “nucleic acid sequence”, “polynucleotide”, “oligonucleotide”, “polynucleotide sequence” and “nucleotide sequence”, used interchangeably in the present description, mean a precise sequence of nucleotides, modified or not, determining a fragment or a region of a nucleic acid, containing unnatural nucleotides or not, and being either a double-strand DNA or RNA, a single-strand DNA or RNA, or transcription products of said DNAs.

Unless otherwise indicated, the term nucleotide sequence encompasses its complement. Thus, a nucleic acid having a particular sequence should be understood as one which encompasses the complementary strand thereof with the complementary sequence thereof.

An “isolated” nucleic acid molecule is one which is identified and separated from at least one nucleic acid molecule-impurity. An isolated nucleic acid molecule is different from the form or set in which it is found under natural conditions. Thus, an isolated nucleic acid molecule is different from a nucleic acid molecule that exists in cells under natural conditions.

In one aspect, the present invention relates to a nucleic acid molecule comprising a nucleotide sequence encoding an amino acid sequence selected from SEQ ID NOs: 1-73. A nucleic acid molecule may also comprise any combination of said nucleotide sequences.

As would be appreciated by those skilled in the art, because of the redundancy of the genetic code, a variety of different DNA sequences can encode the amino acid sequence of the light chain or heavy chain of the antibody according to the invention or fragments thereof (VH, VL, CDR, etc.). It is well within the skill of those trained in the art to create these alternative DNA sequences encoding one and the same amino acid sequences. Such variant DNA sequences are within the scope of the present invention.

In some embodiments of the invention, the isolated nucleic acid is DNA.

The nucleic acid molecule of the invention may be isolated from any source that produces the monoclonal antibody or antigen-binding fragment thereof that specifically binds to BCMA. In certain embodiments of the invention, the nucleic acid molecule of the invention may be synthesized by way of chemical synthesis, rather than isolated.

In some embodiments of the invention, the nucleic acid is a nucleic acid that encodes the amino acid sequence of the heavy chain variable domain of antibody 01-001, and includes the nucleotide sequence with SEQ ID NO: 74.

In some embodiments of the invention, the nucleic acid is a nucleic acid that encodes the amino acid sequence of the light chain variable domain of antibody 01-001, and includes the nucleotide sequence with SEQ ID NO: 82.

In some embodiments of the invention, the nucleic acid is a nucleic acid that encodes the amino acid sequence of the heavy chain of antibody 01-001, and includes the nucleotide sequence with SEQ ID NO: 93.

In some embodiments of the invention, the nucleic acid is a nucleic acid that encodes the amino acid sequence of the light chain of antibody 01-001, and includes the nucleotide sequence with SEQ ID NO: 101.

In some embodiments of the invention, the nucleic acid is a nucleic acid that encodes the amino acid sequence of the heavy chain variable domain of antibody 01-006, and includes the nucleotide sequence with SEQ ID NO: 75.

In some embodiments of the invention, the nucleic acid is a nucleic acid that encodes the amino acid sequence of the light chain variable domain of antibody 01-006, and includes the nucleotide sequence with SEQ ID NO: 83.

In some embodiments of the invention, the nucleic acid is a nucleic acid that encodes the amino acid sequence of the heavy chain of antibody 01-006, and includes the nucleotide sequence with SEQ ID NO: 94.

In some embodiments of the invention, the nucleic acid is a nucleic acid that encodes the amino acid sequence of the light chain of antibody 01-006, and includes the nucleotide sequence with SEQ ID NO: 102.

In some embodiments of the invention, the nucleic acid is a nucleic acid that encodes the amino acid sequence of the heavy chain variable domain of antibody 01-016, and includes the nucleotide sequence with SEQ ID NO: 76.

In some embodiments of the invention, the nucleic acid is a nucleic acid that encodes the amino acid sequence of the light chain variable domain of antibody 01-016, and includes the nucleotide sequence with SEQ ID NO: 84.

In some embodiments of the invention, the nucleic acid is a nucleic acid that encodes the amino acid sequence of the heavy chain of antibody 01-016, and includes the nucleotide sequence with SEQ ID NO: 95.

In some embodiments of the invention, the nucleic acid is a nucleic acid that encodes the amino acid sequence of the light chain of antibody 01-016, and includes the nucleotide sequence with SEQ ID NO: 103.

In some embodiments of the invention, the nucleic acid is a nucleic acid that encodes the amino acid sequence of the heavy chain variable domain of antibody 01-021, and includes the nucleotide sequence with SEQ ID NO: 77.

In some embodiments of the invention, the nucleic acid is a nucleic acid that encodes the amino acid sequence of the light chain variable domain of antibody 01-021, and includes the nucleotide sequence with SEQ ID NO: 85.

In some embodiments of the invention, the nucleic acid is a nucleic acid that encodes the amino acid sequence of the heavy chain of antibody 01-021, and includes the nucleotide sequence with SEQ ID NO: 96.

In some embodiments of the invention, the nucleic acid is a nucleic acid that encodes the amino acid sequence of the light chain of antibody 01-021, and includes the nucleotide sequence with SEQ ID NO: 104.

In some embodiments of the invention, the nucleic acid is a nucleic acid that encodes the amino acid sequence of the heavy chain variable domain of antibody 01-022, and includes the nucleotide sequence with SEQ ID NO: 78.

In some embodiments of the invention, the nucleic acid is a nucleic acid that encodes the amino acid sequence of the light chain variable domain of antibody 01-022, and includes the nucleotide sequence with SEQ ID NO: 86.

In some embodiments of the invention, the nucleic acid is a nucleic acid that encodes the amino acid sequence of the heavy chain of antibody 01-022, and includes the nucleotide sequence with SEQ ID NO: 97.

In some embodiments of the invention, the nucleic acid is a nucleic acid that encodes the amino acid sequence of the light chain of antibody 01-022, and includes the nucleotide sequence with SEQ ID NO: 105.

In some embodiments of the invention, the nucleic acid is a nucleic acid that encodes the amino acid sequence of the heavy chain variable domain of antibody 01-024, and includes the nucleotide sequence with SEQ ID NO: 79.

In some embodiments of the invention, the nucleic acid is a nucleic acid that encodes the amino acid sequence of the light chain variable domain of antibody 01-024, and includes the nucleotide sequence with SEQ ID NO: 87.

In some embodiments of the invention, the nucleic acid is a nucleic acid that encodes the amino acid sequence of the heavy chain of antibody 01-024, and includes the nucleotide sequence with SEQ ID NO: 98.

In some embodiments of the invention, the nucleic acid is a nucleic acid that encodes the amino acid sequence of the light chain of antibody 01-024, and includes the nucleotide sequence with SEQ ID NO: 106.

In some embodiments of the invention, the nucleic acid is a nucleic acid that encodes the amino acid sequence of the heavy chain variable domain of antibody 01-025, and includes the nucleotide sequence with SEQ ID NO: 78.

In some embodiments of the invention, the nucleic acid is a nucleic acid that encodes the amino acid sequence of the light chain variable domain of antibody 01-025, and includes the nucleotide sequence with SEQ ID NO: 88.

In some embodiments of the invention, the nucleic acid is a nucleic acid that encodes the amino acid sequence of the heavy chain of antibody 01-025, and includes the nucleotide sequence with SEQ ID NO: 97.

In some embodiments of the invention, the nucleic acid is a nucleic acid that encodes the amino acid sequence of the light chain of antibody 01-025, and includes the nucleotide sequence with SEQ ID NO: 107.

In some embodiments of the invention, the nucleic acid is a nucleic acid that encodes the amino acid sequence of the heavy chain variable domain of antibody 01-026, and includes the nucleotide sequence with SEQ ID NO: 78.

In some embodiments of the invention, the nucleic acid is a nucleic acid that encodes the amino acid sequence of the light chain variable domain of antibody 01-026, and includes the nucleotide sequence with SEQ ID NO: 89.

In some embodiments of the invention, the nucleic acid is a nucleic acid that encodes the amino acid sequence of the heavy chain of antibody 01-026, and includes the nucleotide sequence with SEQ ID NO: 97.

In some embodiments of the invention, the nucleic acid is a nucleic acid that encodes the amino acid sequence of the light chain of antibody 01-026, and includes the nucleotide sequence with SEQ ID NO: 108.

In some embodiments of the invention, the nucleic acid is a nucleic acid that encodes the amino acid sequence of the heavy chain variable domain of antibody 01-027, and includes the nucleotide sequence with SEQ ID NO: 78.

In some embodiments of the invention, the nucleic acid is a nucleic acid that encodes the amino acid sequence of the light chain variable domain of antibody 01-027, and includes the nucleotide sequence with SEQ ID NO: 87.

In some embodiments of the invention, the nucleic acid is a nucleic acid that encodes the amino acid sequence of the heavy chain of antibody 01-027, and includes the nucleotide sequence with SEQ ID NO: 97.

In some embodiments of the invention, the nucleic acid is a nucleic acid that encodes the amino acid sequence of the light chain of antibody 01-027, and includes the nucleotide sequence with SEQ ID NO: 106.

In some embodiments of the invention, the nucleic acid is a nucleic acid that encodes the amino acid sequence of the heavy chain variable domain of antibody 01-030, and includes the nucleotide sequence with SEQ ID NO: 80.

In some embodiments of the invention, the nucleic acid is a nucleic acid that encodes the amino acid sequence of the light chain variable domain of antibody 01-030, and includes the nucleotide sequence with SEQ ID NO: 90.

In some embodiments of the invention, the nucleic acid is a nucleic acid that encodes the amino acid sequence of the heavy chain of antibody 01-030, and includes the nucleotide sequence with SEQ ID NO: 99.

In some embodiments of the invention, the nucleic acid is a nucleic acid that encodes the amino acid sequence of the light chain of antibody 01-030, and includes the nucleotide sequence with SEQ ID NO: 109.

In some embodiments of the invention, the nucleic acid is a nucleic acid that encodes the amino acid sequence of the heavy chain variable domain of antibody 01-033, and includes the nucleotide sequence with SEQ ID NO: 81.

In some embodiments of the invention, the nucleic acid is a nucleic acid that encodes the amino acid sequence of the light chain variable domain of antibody 01-033, and includes the nucleotide sequence with SEQ ID NO: 91.

In some embodiments of the invention, the nucleic acid is a nucleic acid that encodes the amino acid sequence of the heavy chain of antibody 01-033, and includes the nucleotide sequence with SEQ ID NO: 100.

In some embodiments of the invention, the nucleic acid is a nucleic acid that encodes the amino acid sequence of the light chain of antibody 01-033, and includes the nucleotide sequence with SEQ ID NO: 110.

In some embodiments of the invention, the nucleic acid is a nucleic acid that encodes the amino acid sequence of the heavy chain variable domain of antibody 01-037, and includes the nucleotide sequence with SEQ ID NO: 78.

In some embodiments of the invention, the nucleic acid is a nucleic acid that encodes the amino acid sequence of the light chain variable domain of antibody 01-037, and includes the nucleotide sequence with SEQ ID NO: 92.

In some embodiments of the invention, the nucleic acid is a nucleic acid that encodes the amino acid sequence of the heavy chain of antibody 01-037, and includes the nucleotide sequence with SEQ ID NO: 97.

In some embodiments of the invention, the nucleic acid is a nucleic acid that encodes the amino acid sequence of the light chain of antibody 01-037, and includes the nucleotide sequence with SEQ ID NO: 111.

The nucleic acid molecules may be used to express the monoclonal antibody or antigen-binding fragment thereof that specifically binds to BCMA.

### Vector

In one aspect, the present invention relates to an expression vector comprising any one of the above nucleic acid molecules that encode the corresponding amino acid sequences of the antibody that specifically binds to BCMA, or portions thereof (for example, heavy chain and/or light chain binding domain sequences). The present invention relates to a vector suitable for the expression of any one of nucleotide sequences described herein.

The term “vector” as used herein means a nucleic acid molecule capable of transporting another nucleic acid to which it has been linked.

As used in the present description, the term “expression” is defined as the transcription and/or translation of a particular nucleotide sequence driven by its promoter.

In some embodiments of the invention, the vector is a plasmid, i.e. a circular double stranded piece of DNA into which additional DNA segments may be inserted.

In some embodiments of the invention, the vector is a viral (expression) vector, wherein additional DNA segments may be inserted into the viral genome.

In some embodiments of the invention, the vectors are capable of autonomous replication in a host cell into which they are introduced (e.g. bacterial vectors having a bacterial site of replication origin and episomal vectors). In further embodiments of the invention, vectors (e.g. non-episomal vectors) may be integrated into the genome of a host cell upon introduction into a host cell, and thereby are replicated along with the host gene. Moreover, certain vectors are capable of directing the expression of genes to which they are operably linked. Such vectors are referred to herein as “recombinant expression vectors” (or simply, “expression vectors”).

In some embodiments of the invention, expression vectors include plasmids, retroviruses, adenoviruses, adeno-associated viruses (AAVs), plant viruses, such as cauliflower mosaic virus, tobacco mosaic virus, cosmids, YACs, and the like. DNA molecules may be inserted into a vector such that transcriptional and translational control sequences within the vector serve their intended function of regulating the transcription and translation of DNA. An expression vector and expression control sequences may be chosen to be compatible with the expression host cell used.

In one embodiment of the invention, DNA molecules partially or fully encoding the sequences of first and second binding domains (for example, heavy and light chain sequences where a binding domain comprises a heavy and light chain sequence) may be introduced into individual vectors.

In one embodiment, any combination of the above DNA molecules is introduced into the same expression vector.

In one embodiment of the invention, DNA molecules may be introduced into an expression vector by standard methods (e.g. ligation of complementary restriction sites on a gene fragment of antibody and vector, or blunt end ligation if no restriction sites are present).

In some embodiments of the invention, a suitable vector is one that includes restriction sites such that any VH or VL sequence can easily be inserted and expressed, as described above. Polyadenylation and transcription termination may occur at a native chromosomal site downstream of coding regions. A recombinant expression vector can also encode a signal peptide that facilitates secretion of an antibody chain from a host cell. An antibody chain gene may be cloned into a vector such that the signal peptide is linked in-frame to the amino terminus of an immunoglobulin chain. A signal peptide may be an immunoglobulin signal peptide or a heterologous signal peptide (i.e. a signal peptide from a non-immunoglobulin protein).

In some embodiments of the invention, the vector may include an expression control sequence. The term “expression control sequence” as used in the present description refers to polynucleotide sequences that are necessary to effect the expression and processing of coding sequences to which they are inserted. It will be understood by those skilled in the art that the design of an expression vector, including the selection of expression control sequences, may depend on such factors as the choice of the type of a host cell to be transformed, the required level of expression of antibody, and so forth. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such expression control sequences differs depending upon the host organism; in prokaryotes, such expression control sequences typically include a promoter, a ribosome binding site, as well as transcription termination sequences; in eukaryotes, such expression control sequences typically include promoters and transcription termination sequences. Preferred expression control sequences for an expression host cell in a mammal include viral elements that ensure high levels of protein expression in mammalian cells, such as promoters and/or enhancers derived from a retroviral LTR, cytomegalovirus (CMV) (such as a CMV promoter/enhancer), simian virus 40 (SV40) (such as a SV40 promoter/enhancer), adenovirus, (e.g. the major late promoter adenovirus (AdMLP)), polyomavirus and strong mammalian promoters such as TTR promoter, native immunoglobulin promoter or actin promoter. Expression control sequences encompass at least all components whose presence is important for expression and processing.

In some embodiments of the invention, in addition to antibody chain genes and expression control sequences, the recombinant expression vectors of the invention may carry additional sequences, such as sequences that regulate replication of a vector in host cells (e.g. origins of replication) and selectable marker genes. The selectable marker gene facilitates the selection of host cells into which a vector has been introduced.

### Host Cell

In one aspect, the present invention relates to a method for producing a host cell to produce any above antibody or antigen-binding fragment thereof that specifically binds to BCMA, and includes transformation of the cell with the above vector.

In one aspect, the present invention relates to a host cell for producing any above antibody or antigen-binding fragment thereof that specifically binds to BCMA, comprising any one of the above nucleic acids.

The term “host cell” as used herein refers to a cell into which a recombinant expression vector has been introduced. The present invention relates to host cells, which may include, for example, the above-described vector according to the invention. The present invention further relates to host cells that comprise, for example, a nucleotide sequence encoding a heavy chain or antigen-binding portions thereof, a nucleotide sequence encoding a light chain or antigen-binding portions thereof, or both. It should be understood that “host cell” refers not only to a particular subject cell but to the progeny of such cell as well. Since modifications may occur in succeeding generations due to either mutation or environmental influences, such progeny may not, in fact, be identical to a parental cell; however, such cells are still included within the scope of the term “host cell” as used herein.

Nucleic acid molecules encoding the monoclonal antibody or antigen-binding fragment thereof that specifically binds to BCMA according to the invention and vectors comprising these nucleic acid molecules may be used for transfection of a mammalian cell, plant cell, bacterial cell, or yeast cell. Transfection may be carried out by any known method for introducing polynucleotides into a host cell. Methods for introducing heterologous polynucleotides into mammalian cells are well known in the art and include dextran-mediated transfection, cationic polymer-nucleic acid complex transfection, calcium phosphate precipitation, polybrene-mediated transfection, protoplast fusion, encapsulation of the polynucleotides in liposomes, and direct microinjection of DNA into nuclei. In addition, the nucleic acid molecules may be introduced into mammalian cells by viral (expression) vectors.

Mammalian cell lines used as hosts for transformation are well known in the art and include a plurality of immortalized cell lines available. These include, e.g., Chinese hamster ovary (CHO) cells, NS0 cells, SP2 cells, HEK-293T cells, FreeStyle 293 cells (Invitrogen), NIH-3T3 cells, HeLa cells, baby hamster kidney (BHK) cells, African green monkey kidney cells (COS), human hepatocellular carcinoma cells (e.g., Hep G2), A549, SK-HEP1, HUH7, Hep-RG cells and a number of other cell lines. Cell lines are selected by way of determining which cell lines have high expression levels and provide for necessary characteristics of the protein being produced. Other cell lines that may be used are insect cell lines, such as Sf9 or Sf21 cells. When recombinant expression vectors encoding the monoclonal antibody or antigen-binding fragment thereof that specifically binds to BCMA are introduced into mammalian host cells, the antibodies or fragments thereof are produced by culturing the host cells for a period of time sufficient to allow for expression of the antibodies or fragments thereof in host cells or, more preferably, secretion of the antibodies or fragments thereof into the culture medium in which the host cells are grown. The monoclonal antibody or antigen-binding fragment thereof that specifically binds to BCMA may be isolated from culture medium using standard protein purification techniques. Plant host cells include e.g. Nicotiana, Arabidopsis, duckweed, corn, wheat, potato, etc. Bacterial host cells include Escherichia and Streptomyces species. Yeast host cells include Schizosaccharomyces pombe, Saccharomyces cerevisiae and Pichia pastoris.

Furthermore, level of production of the monoclonal antibody or antigen-binding fragment thereof that specifically binds to BCMA from a production cell line may be enhanced using a number of known techniques. For example, the glutamine synthetase gene expression system (the GS system) is a common approach for enhancing expression under certain conditions.

It is likely that the monoclonal antibody or antigen-binding fragment thereof that specifically binds to BCMA from various cell lines will have a different glycosylation profile as compared to one another. However, the monoclonal antibody or antigen-binding fragment thereof that specifically binds to BCMA encoded by nucleic acid molecules described herein, or comprising amino acid sequences provided herein are part of the present invention, regardless of the glycosylation of the binding molecules, and, in general, regardless of the presence or absence of post-translational modifications.

The above host cell does not relate to a host cell produced using human embryos.

The above host cell does not relate to a host cell produced by modifying the genetic integrity of human germline cells.

### Method for Producing Antibody

In one aspect, the present invention relates to a method for producing an antibody or antigen-binding fragment thereof that specifically binds to BCMA, comprising culturing the above host cell in a growth medium under conditions sufficient to produce said antibody or fragment thereof, if necessary, followed by isolation and purification of the resulting antibody or fragment thereof.

### Pharmaceutical Compositions

Another aspect of the invention is a pharmaceutical composition comprising, as an active ingredient (or as the only active ingredient), the monoclonal antibody according to the present invention or antigen-binding fragment thereof that specifically binds to BCMA.

In one aspect, the present invention relates to a pharmaceutical composition used for treating a BCMA-mediated disease or disorder, which comprises any above antibody or antigen-binding fragment thereof in a therapeutically effective amount in combination with one or more pharmaceutically acceptable excipients.

“Pharmaceutical composition” means a composition comprising an antibody according to the invention and at least one of components selected from the group consisting of pharmaceutically acceptable and pharmacologically compatible fillers, solvents, diluents, carriers, auxiliary, distributing and sensing agents, delivery agents.

The term “pharmaceutically acceptable” refers to one or more compatible liquid or solid components that are suitable for administration in a mammal, preferably in a human.

The term “excipient” is used herein to describe any ingredient other than the antibody according to the present invention. These are substances of inorganic or organic nature which are used in the pharmaceutical production/manufacturing in order to give drug products the necessary physicochemical properties.

In some embodiments, the compositions are intended to improve, prevent, or treat diseases or disorders that may be mediated by BCMA.

The term “BCMA-mediated disease or disorder” refers to any disease or disorder that is either directly, or indirectly associated with BCMA, including etiology, development, progression, persistence or pathology of a disease or disorder.

“Treat”, “treatment” and “therapy” refer to a method of alleviating or abrogating a biological disorder and/or at least one of attendant symptoms thereof. Further, references herein to “treatment” include references to curative, palliative and prophylactic treatment.

The term “disorder” means any condition that would benefit from treatment according to the present invention. The definition of the term includes chronic and acute disorders or diseases including those pathological conditions that predispose the mammal to the disorder in question.

“Therapeutically effective amount” refers to that amount of the therapeutic agent being administered during treatment which will relieve to some extent one or more of the symptoms of the disease being treated. A therapeutically effective amount may vary according to factors such as the particular condition being treated, the age, sex and weight of the patient, and whether the monoclonal antibody or antigen-binding fragment thereof that specifically binds to BCMA is being administered as a stand-alone treatment or in combination with one or more additional drugs or treatments.

In one aspect, the subject of treatment, or patient, is a mammal, preferably a human subject. Said subject may be either male or female, of any age.

The pharmaceutical compositions of the present invention and methods of preparation thereof will be undoubtedly apparent to those skilled in the art. The pharmaceutical compositions should preferably be manufactured in compliance with the GMP (Good Manufacturing Practice) requirements.

In some embodiments of the pharmaceutical composition, it may include a buffer composition, tonicity agents (osmolyte or osmotic agent), stabilizers and/or solubilizers.

The pharmaceutical composition according to the invention is a stable composition.

A pharmaceutical composition is “stable” if the active agent retains physical stability and/or chemical stability and/or biological activity thereof during the specified shelf life at storage temperature, for example, of 2-8° C. Preferably, the active agent retains both physical and chemical stability, as well as biological activity. Storage period is adjusted based on the results of stability test in accelerated or natural aging conditions.

The pharmaceutical composition according to the present invention is suitable for parenteral administration as sterile formulations intended for administration in a subject body through the breach in skin or mucosal barriers, bypassing the gastrointestinal tract by virtue of injection, infusion and implantation. In particular, it is contemplated that parenteral administration includes, inter alia, subcutaneous, intraperitoneal, intramuscular, intravenous, intraarterial, intrathecal, intraventricular, intraurethral, intracranial, intrasynovial, transdermal injection or infusion; and kidney dialytic infusion techniques. Intra-tumor delivery, for example, intra-tumor injection, may also be employed. Regional perfusion is also contemplated.

In some embodiments, the pharmaceutical composition is administered intravenously.

In some embodiments, intravenous administration is carried out by using infusion, prolonged infusion, or long-lasting continuous infusion.

In some embodiments, the pharmaceutical composition is administered subcutaneously.

In some embodiments, subcutaneous administration is carried out by using subcutaneous injection.

In some embodiments, the pharmaceutical composition is an injectable dosage form.

In some embodiments, the injectable dosage form is an infusion solution.

In some embodiments, the injectable dosage form is a solution for subcutaneous administration.

Injectable formulations may be manufactured without limitation, in unit dosage form, such as in ampoules, vials, plastic containers, pre-filled syringes, autoinjection devices.

In some embodiments, the pharmaceutical composition is a pharmaceutical composition provided in dry, i.e. powder or granular, form for reconstitution with a suitable solvent (e.g., sterile pyrogen-free water) prior to administration. Such medicinal formulation may be prepared by, for example, lyophilization, i.e. a process, which is known in the art as freeze drying, and which involves freezing a product followed by removal of solvent from frozen material.

In some embodiments, the pharmaceutical composition is a lyophilizate for preparing a solution for infusion.

In some embodiments, the pharmaceutical composition is a lyophilizate for preparing a solution for subcutaneous administration.

In some embodiments, the pharmaceutical composition is a concentrate for preparing a solution for infusion.

In some embodiments, the pharmaceutical composition is a concentrate for preparing a solution for subcutaneous administration.

In one aspect, the present invention relates to a pharmaceutical composition that comprises a monoclonal antibody according to the present invention or antigen-binding fragment thereof that specifically binds to BCMA and at least one other therapeutically active compound.

In one aspect, the present invention relates to a pharmaceutical composition for treating a BCMA-mediated disease or disorder, comprising any above antibody or antigen-binding fragment thereof and at least one other therapeutically active compound.

In one aspect, the present invention relates to a pharmaceutical composition comprising any above antibody or antigen-binding fragment thereof and further at least one other therapeutically active compound.

In one aspect, the present invention relates to a pharmaceutical composition for treating a BCMA-mediated disease or disorder, comprising any above antibody or antigen-binding fragment thereof and further at least one other therapeutically active compound.

In one aspect, the present invention relates to a pharmaceutical composition for treating a BCMA-mediated disease or disorder, comprising any above antibody or antigen-binding fragment thereof and at least one other therapeutically active compound, which is an antibody, a small molecule, a hormone therapy agent or a combination thereof.

In some embodiments of the pharmaceutical composition, the BCMA-mediated disease or disorder is selected from the group: multiple myeloma, chronic lymphocytic leukemia, B-cell acute lymphoblastic leukemia, non-Hodgkin's lymphoma, Hodgkin's lymphoma.

Therapeutic Use of Monoclonal Antibody or Antigen-Binding Fragment Thereof that Specifically Binds to BCMA

In one aspect, the antibody or antigen-binding fragment thereof that specifically binds to BCMA is used in the treatment of disorders mediated by BCMA activity.

In one aspect, the subject of treatment, or patient, is a mammal, preferably a human subject. Said subject may be either male or female, of any age.

In one aspect, the present invention relates to a method for treating a BCMA-mediated disease or disorder, comprising administering in a subject in need of such treatment any above antibody or antigen-binding fragment thereof or said pharmaceutical composition, in a therapeutically effective amount.

In one aspect, the present invention relates to a method for treating a BCMA-mediated disease or disorder, comprising administering in a subject in need of such treatment any above antibody or antigen-binding fragment thereof and at least one other therapeutically active compound in a therapeutically effective amount.

In some embodiments of the method for treating, the BCMA-mediated disease or disorder is selected from the group: multiple myeloma, chronic lymphocytic leukemia, B-cell acute lymphoblastic leukemia, non-Hodgkin's lymphoma, Hodgkin's lymphoma.

In some embodiments of the method for treating, the other therapeutically active compound is a small molecule, hormone therapy agent, or any combination thereof.

In one aspect, the present invention relates to the use of the above antibody or antigen-binding fragment thereof or the above pharmaceutical composition for treating in a subject in need of such treatment a BCMA-mediated disease or disorder.

In one aspect, the present invention relates to the use of the above antibody or antigen-binding fragment thereof and at least one other therapeutically active compound for treating a BCMA-mediated disease or disorder.

In some embodiments of the use, the BCMA-mediated disease or disorder is selected from the group: multiple myeloma, chronic lymphocytic leukemia, B-cell acute lymphoblastic leukemia, non-Hodgkin's lymphoma, Hodgkin's lymphoma.

In some embodiments of the use, the other therapeutically active compound is a small molecule, hormone therapy agent, or any combination thereof.

The uses or methods used herein relating to the antibody or antigen-binding fragment thereof that specifically binds to BCMA with one or more other therapeutic agents are contemplated to mean, refer to and include the following:
1) simultaneous administration of such combination of the antibody or antigen-binding fragment thereof that specifically binds to BCMA and therapeutic agent to a patient in need of treatment, when such components are formulated together into a single dosage form which releases said components at substantially the same time to said patient,
2) simultaneous administration of such combination of the antibody or antigen-binding fragment thereof that specifically binds to BCMA and therapeutic agent to a patient in need of treatment, when such components are formulated apart from each other into separate dosage forms which are taken at substantially the same time by said patient, whereupon said components are released at substantially the same time to said patient,
3) sequential administration of such combination of the antibody or antigen-binding fragment thereof that specifically binds to BCMA and therapeutic agent to a patient in need of treatment, when such components are formulated apart from each other into separate dosage forms which are taken at consecutive times by said patient with a significant time interval between each administration, whereupon said components are released at substantially different times to said patient; and
4) sequential administration of such combination of the antibody or antigen-binding fragment thereof that specifically binds to BCMA and therapeutic agent to a patient in need of treatment, when such components are formulated together into a single dosage form which releases said components in a controlled manner, whereupon they are concurrently, consecutively, or jointly released at the same and/or different times to said patient, where each portion may be administered by either the same or different routes.

The antibody or antigen-binding fragment thereof that specifically binds to BCMA may be administered without further therapeutic treatment, i.e. as an independent therapy.

In some embodiments of the method for treating or the use, the antibody or antigen-binding fragment that specifically binds to BCMA may be administered in combination with proteasome inhibitors.

In some embodiments of the method for treating or the use, the antibody or antigen-binding fragment that specifically binds to BCMA may be administered in combination with antitumor immunomodulators (for example, lenalidomide, pomalidomide).

In some embodiments of the method for treating or the use, the antibody or antigen-binding fragment that specifically binds to BCMA may be administered in combination with cytostatic chemotherapy (cyclophosphamide, etoposide, and the like).

In some embodiments of the method for treating or the use, the antibody or antigen-binding fragment that specifically binds to BCMA may be administered in combination with tyrosine kinase inhibitors.

In some embodiments of the method for treating or the use, the antibody or antigen-binding fragment that specifically binds to BCMA may be administered in combination with BCL2 inhibitor products.

In some embodiments of the method for treating or the use, the antibody or antigen-binding fragment that specifically binds to BCMA may be administered in combination with exportin 1 antagonist products.

In some embodiments of the method for treating or the use, the antibody or antigen-binding fragment that specifically binds to BCMA may be administered in combination with glucocorticosteroids.

In some embodiments of the method for treating or the use, the antibody or antigen-binding fragment that specifically binds to BCMA may be administered in combination with antitumor monoclonal antibodies (for example, daratumumab).

In some embodiments of the method for treating or the use, the antibody or antigen-binding fragment that specifically binds to BCMA may be administered in combination with targeted therapy.

In some embodiments of the method for treating or the use, the antibody or antigen-binding fragment that specifically binds to BCMA may be administered in combination with monoclonal antibodies agonistic to cytokines (e.g., IL15SA, IL2).

In some embodiments of the method for treating or the use, the antibody or antigen-binding fragment that specifically binds to BCMA may be administered in combination with monoclonal antibodies antagonistic to cytokines (e.g., anti-IL6R, anti-TNF).

In some embodiments of the method for treating or the use, the antibody or antigen-binding fragment that specifically binds to BCMA may be administered in combination with G-CSF (granulocyte colony-stimulating factor) products.

In some embodiments of the method for treating or the use, the antibody or antigen-binding fragment that specifically binds to BCMA may be administered in complex with hematopoietic stem cell transplantation.

In some embodiments of the method for treating or the use, the antibody or antigen-binding fragment that specifically binds to BCMA may be administered in complex with radiation therapy.

In some embodiments of the method for treating or the use, a suitable dose of the monoclonal antibody or antigen-binding fragment thereof that specifically binds to BCMA according to the present invention will range from 0.1 to 200 mg/kg.

### BRIEF DESCRIPTION OF DRAWINGS

FIG. 1 is a map of an expression vector bearing a genetic sequence of variable domains.

FIG. 2 is a map of an expression vector bearing a genetic sequence of variable domains.

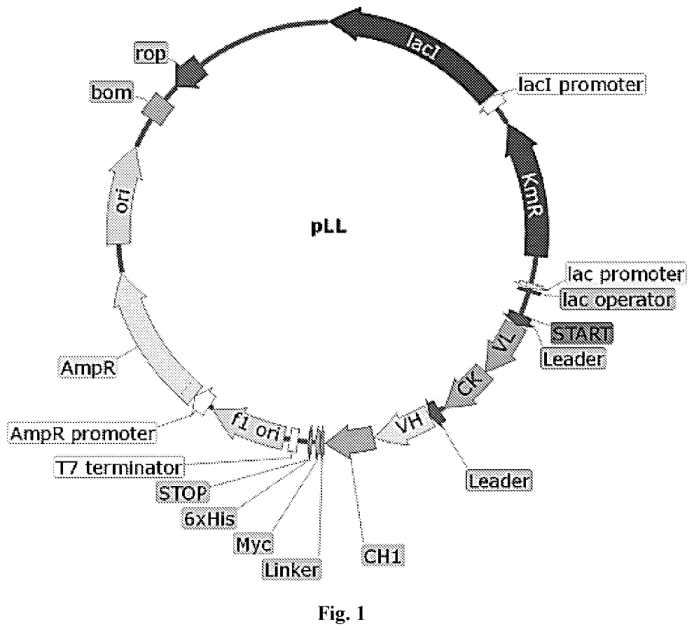

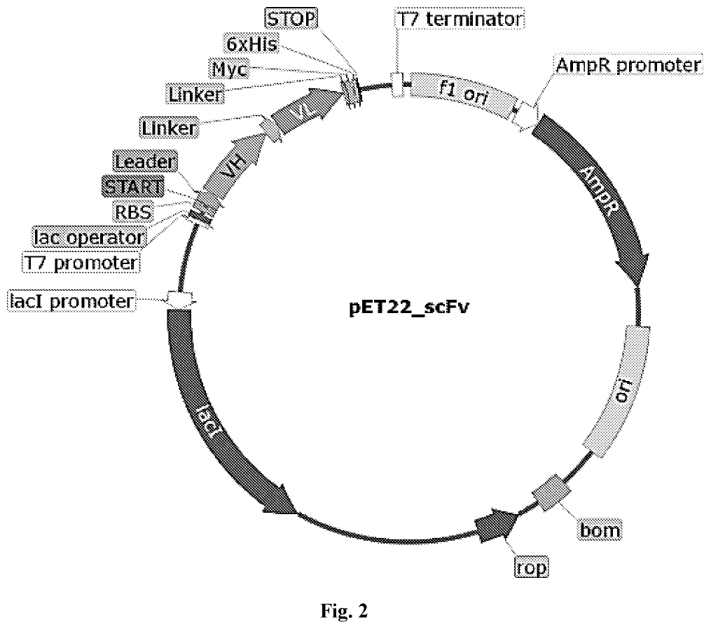

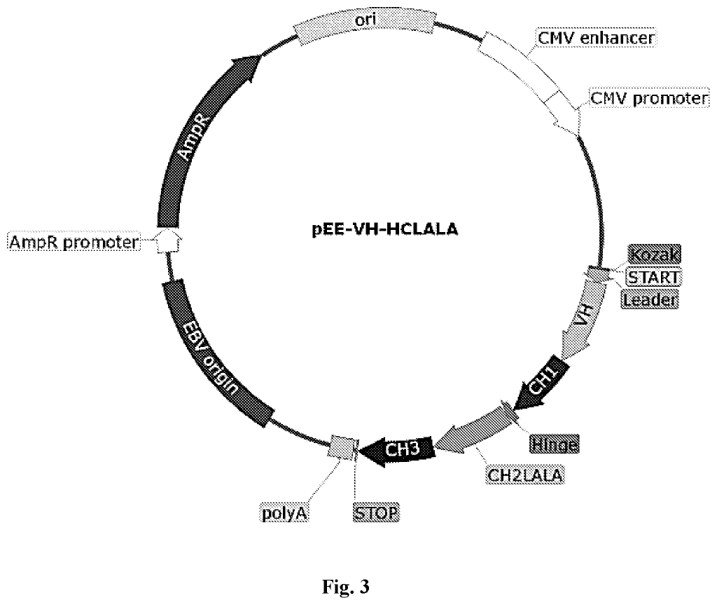

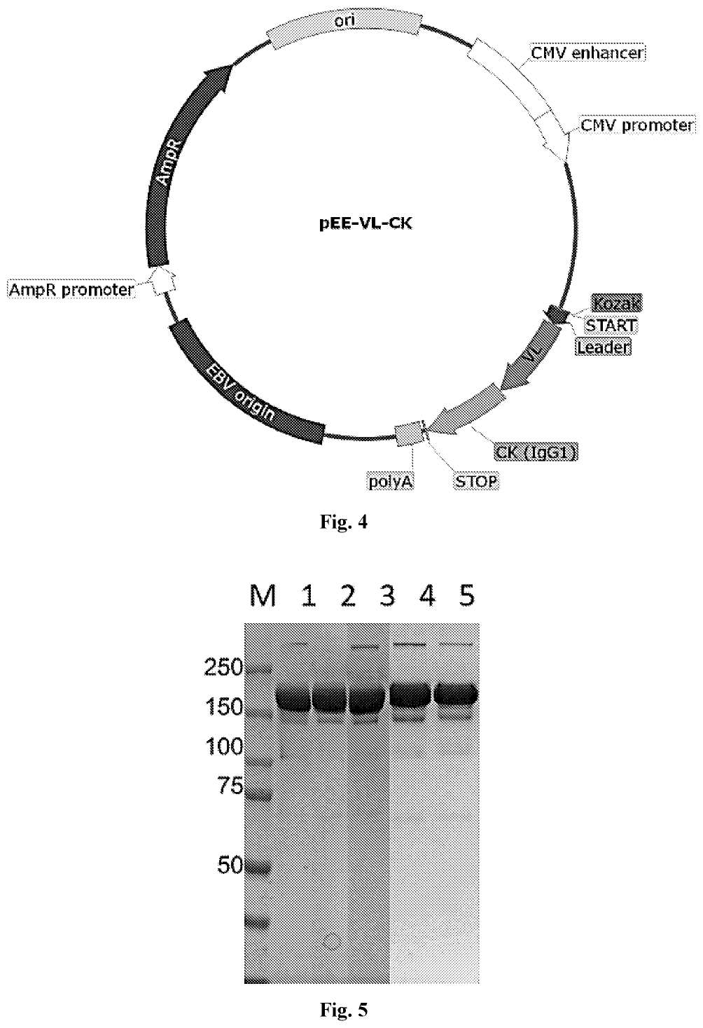

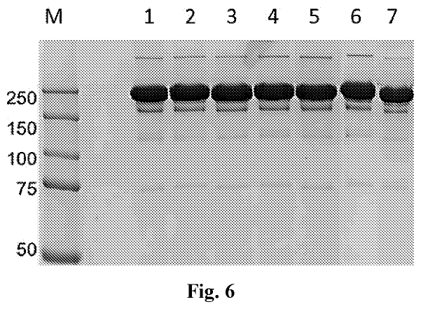

For FIGS. 1 to 2

| Name | Definition |
|---|---|
| HpelB/LpelB/leader | Signal leader peptide |
| VH | Antibody heavy chain variable domain gene. |
| Vκ/VL | Antibody light chain variable domain gene. |
| CK | CK (kappa) light chain constant domain gene |
| CH1 | Gene of first constant domain of human IgG1 |
| | heavy chain. |
| 6xHis | Histidine sequence |
| Myc | Myc tag |
| STOP-LC, STOP-HC, | Stop codon |
| STOP | |
| START | Start codon |
| ori | Replication origin. Functions in E. coli cells |
| AmpR | Beta-lactamase gene that confers resistance to |
| | ampicillin. Enables selection in E. coli cell |
| | culture |
| KmR | Kanamycin resistance gene |
| lacI | Lactose repressor |
| linker | Linker |
| RBS | Ribosome binding site |
| T7 promoter | T7 promoter |
| T7 terminator | T7 terminator |
| AmpR promoter | AmpR promoter |
| lacI operator | Lactose operon operator |
| lacI promoter | Lactose operon promoter |
| Rop | Regulatory protein Rop |
| f1_ori | Origin of bacteriophage f1 DNA replication |
| Bom | Region responsible for plasmid transfer in the |
| | presence of conjugation plasmid. |

FIG. 3 is a map of vector bearing the genetic sequence of antibody heavy chain.

FIG. 4 is a map of vector bearing the genetic sequence of antibody light chain.

For FIGS. 3 to 4

| Name | Definition |
|---|---|
| CMV-promotor | Eukaryotic CMV promoter |
| Leader | Murine IgK signal leader peptide |
| VH | Antibody heavy chain variable domain gene. |
| VL | Antibody light chain variable domain gene. |
| CK | CK (kappa) light chain constant domain gene. |
| CH1 | CH1 constant domain of human IgG1 heavy |
| | chain |
| CH2LALA | CH2 domain of human IgG1 heavy chain with |
| | L234A and L235A mutations |
| CH3 | CH3 constant domain of human IgG1 heavy |
| | chain |
| STOP | Stop codon |
| PolyA | Polyadenylation signal |
| EBV origin | Eukaryotic origin of Epstein-Barr virus |
| | replication |
| Hinge | Human IgG1 hinge region |
| AmpR | Beta-lactamase gene that confers resistance to |
| | ampicillin. Enables selection in E. coli cell |
| | culture |
| CMV_enhancer | Cytomegalovirus enhancer |
| Kozak | Kozak consensus sequence |
| START | Start codon |
| AmpR promoter | Beta-lactamase gene promoter |
| Ori | High-copy bacterial origin of replication |

FIG. 5 is an electrophoregram of candidates 01, 06, 016, 021 and 022 in 7.5% polyacrylamide gel under denaturing non-reducing conditions following the first stage of purification on Protein A sorbent.

M is molecular weight marker.

1—candidate 01.

2—candidate 06.

3—candidate 016.

4—candidate 021.

5—candidate 022

FIG. 6 is an electrophoregram of candidates 024, 025, 026, 027, 030, 033 and 037 in 7.5% polyacrylamide gel under denaturing non-reducing conditions following the first stage of purification on Protein A sorbent.

M is molecular weight marker.

1—candidate 024.

2—candidate 025.

3—candidate 026.

4—candidate 027.

5—candidate 030.

6—candidate 033.

7—candidate 037.

### EXAMPLES

The following examples are provided for better understanding of the invention. These examples are for purposes of illustration only and are not to be construed as limiting the scope of the invention in any manner.

All publications, patents, and patent applications cited in this specification are incorporated herein by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended embodiments.

### Materials and General Methods

General information regarding the nucleotide sequences of human immunoglobulin light and heavy chains is given in: Kabat, E. A., et al., Sequences of Proteins of Immunological Interest, 5th ed., Public Health Service, National Institutes of Health, Bethesda, MD (1991). Amino acids of antibody chains are numbered according to EU numbering (Edelman, G. M., et al., Proc. Natl. Acad. Sci. USA 63 (1969) 78-85; Kabat, E. A., et al., Sequences of Proteins of Immunological Interest, 5th ed., Public Health Service, National Institutes of Health, Bethesda, MD, (1991).

### Recombinant DNA Techniques

Standard methods were used to manipulate DNA as described in Sambrook, J. et al, Molecular cloning: A laboratory manual; Cold Spring Harbor Laboratory Press, Cold Spring Harbor, New York, 1989. The molecular biological reagents were used according to the manufacturer protocols.

### Gene Synthesis

Desired gene segments were prepared from oligonucleotides made by chemical synthesis. The gene segments of 300-1400 bp long, which were flanked by singular restriction sites, were assembled by annealing and ligation of oligonucleotides including PCR amplification and subsequently cloned via the restriction sites. The DNA sequences of the subcloned gene fragments were confirmed by DNA sequencing.

### DNA Sequence Determination

DNA sequences were determined by Sanger sequencing.

### DNA and Protein Sequence Analysis and Sequence Data Management

The Unipro's UGENE suite version 1.29 and SnapGene Viewer were used for sequence creation, mapping, analysis, annotation and illustration.

### Expression Vectors

For the expression of the antibodies described in the application materials, variants of expression plasmids intended for expression of antibodies in prokaryotic cells (E. coli), transient expression in eukaryotic cells (e.g., in CHO cells) were applied. Beside the antibody expression cassette, the vectors contained: an origin of replication which allows replication of said plasmid in E. coli, genes which confer resistance in E. coli to various antibiotics (e.g. to ampicillin, kanamycin).

The fusion genes comprising the described antibody chains as described below were generated by PCR and/or gene synthesis and assembled with known recombinant methods and techniques by connection of the according nucleic acid segments, e.g. using unique restriction sites in the corresponding vectors. The subcloned nucleic acid sequences were verified by DNA sequencing. For transient transfections, larger quantities of the plasmids were prepared by plasmid preparation from transformed E. coli cultures.

### Example 1. Production of Recombinant Antigens in Suspension Culture of Mammalian Cells

Sequences of extracellular domains of the human BCMA receptor (SEQ https://www.uniprot.org/uniprot/Q02223 (1-54 aa)) were cloned into pEE plasmids for protein generation in mammalian cells with TEV-Fc, Avi-His, Avi-His-TEV-HSA tags via SalI/NotI restriction sites. The required quantities of plasmids were cultured in E. Coli cells and purified using the Qiagen kit.

Antigens were produced in established cell line obtained from Chinese hamster ovary (CHO-T) cells. Suspension culturing was carried out in flasks on an orbital incubator shaker. To express the target antigens, the cells were transfected using linear polyethylenimine. 9 days following transfection, culture liquid was separated from cells by filtration through a 0.5/0.22 μm deep-bed filter.

The antigen BCMA-TEV-Fc was isolated from the culture fluid and purified using a column with Protein A affinity chromatography sorbent. The cleared culture liquid was passed through a column equilibrated with phosphate buffered saline (PBS, pH 7.4). Bound antigen was eluted using 0.1 M glycine buffer (pH 2.5.). The resulting target protein was then dialyzed into PBS (pH 7.4), DTT was added, the mixture was filtered through 0.22 μm Millex GP, transferred into tubes and stored at −70° C. The BCMA-Avi-His, BCMA-Avi-His-TEV-HSA antigens were isolated from the culture liquid and purified using a Ni-NTA affinity chromatography column. The cleared culture liquid was passed through a column equilibrated with phosphate buffered saline (PBS, pH 7.4). Bound antigen was eluted with PBS supplemented with 500 mM imidazole (pH 7.4). The protein was then dialyzed into PBS. Thereafter, the protein was applied onto a gel filtration column. The resulting protein was filtered through 0.22 μm Millex GP, transferred into tubes and stored at −70° C.

### Example 2. Cloning of Variable Domain Genes of Antibodies 01-001, 01-006, 01-016, 01-021, 01-022, 01-024, 01-025, 01-026, 01-027, 01-030, 01-033, 01-037 into Expression Plasmid

Variable domain genes of antibodies were cloned into expression plasmids pLL (FIG. 1) and pET22 (FIG. 2) according to a standard protocol using the restriction ligation method.

Subsequently, expression vectors comprising antibody fragments were transformed into E. coli BL21-DE3 and BL21-Gold strains for comparative analysis of affinity of variable antibody fragments from display libraries to antigen by ELISA using an automated platform.

### Example 3. Generation and Primary Analysis of Fabs and scFvs of Antibodies 01-001, 01-006, 01-016, 01-021, 01-022, 01-024, 01-025, 01-026, 01-027, 01-030, 01-033, 01-037 Specifically Binding the Human BCMA

Fabs and scFvs were generated according to the standard technique: E. coli BL21-DE3 and BL21-Gold bacterial cells were transformed with expression vectors containing scFv and Fab genes, respectively, and the subsequent addition of an inducer triggered transcription of lac operon, thus, while culturing the resulting transformants, causing expression of scFvs and Fabs which were exported into periplasmic space. ELISA was then performed to verify scFv and Fab binding to substrate-immobilized antigen hBCMA-Avi-His at a concentration of 0.2 μg/ml in 0.1 M NaHCO₃ (pH 9.0) (the antigen was immobilized overnight at 4° C.). All further steps were carried out at room temperature according to the standard ELISA protocol using a high-throughput automated platform based on robotic systems. Washing was performed after each step, with 300 μl/well 1×PBST in three replications. Non-specific binding sites on the plate were blocked with 1% fat-free milk in 1×PBST; analyte (60 μl/well of E. coli supernatants) was added following washing. Immune complexes were detected using peroxidase-conjugated goat anti-Myc-tag antibodies (1:20000). Substrate-chromogenic mixture was stained by adding 50 μl of substrate solution for 15 minutes. 25 μl of 1% H₂SO₄ was used to stop the reaction. The color signal was measured at a wavelength of 450 nm. Antibody binding level was proportional to the signal produced. Clones in which a colour signal exceeded the signal from control antibody were tested in ELISA against non-specific binding.

Thus, all tested Fabs and scFvs of antibodies 01-001, 01-006, 01-016, 01-021, 01-022, 01-024, 01-025, 01-026, 01-027, 01-030, 01-033, 01-037 exhibit specific binding to hBCMA-Avi-His.

### Example 4. Analysis of Nonspecific Binding of Fabs and scFvs of Antibodies 01-001, 01-006, 01-016, 01-021, 01-022, 01-024, 01-025, 01-026, 01-027, 01-030, 01-033, 01-037 to Other Antigens

ELISA was also employed to analyze non-specific binding of scFvs and Fabs in question to different antigens. Analysis was performed as described above, except for the fact that hCD16-Avi-His-FLAG, cynoIL4R-Fc in 0.1 M NaHCO₃ (pH 9.0) were used as antigens for immobilization (antigens were immobilized overnight at 4° C.). hBCMA-Avi-His, rhesusBCMA were used as controls for specific binding (antigens were immobilized overnight at 4° C.). All subsequent steps were carried out according to the standard ELISA protocol using a high-throughput automated platform.

All tested Fabs and scFvs of antibodies 01-001, 01-006, 01-016, 01-021, 01-022, 01-024, 01-025, 01-026, 01-027, 01-030, 01-033, 01-037 exhibit specific binding to hBCMA-Avi-His, rhesusBCMA and do not exhibit non-specific interaction with hCD16-Avi-His-FLAG and cynoIL4R-Fc.

### Example 5. Analysis of Fabs and scFvs of Antibodies 01-001, 01-006, 01-016, 01-021, 01-022, 01-024, 01-025, 01-026, 01-027, 01-030, 01-033, 01-037 in Terms of Dissociation Rate on ForteBio

Fabs were measured on the ForteBio Octet RED 384 system using FAB2G biosensors (ForteBio). Fab samples were loaded onto sensors from supernatants for 16-18 hours at temperature conditions of (5±3) ° C. When loaded, the sensors were transferred to a kinetic buffer solution with 500 mM NaCl (4.3 mM Na₂HPO₄; 500 mM NaCl, 1.5 mM KH₂PO₄; 2.7 mM KCl; the volume fraction of Tween 20 added was 0.1%; the mass fraction of BSA added was 0.1%; pH 7.4), the given buffer solution was used for preparing all subsequent steps of experiments involving Fab samples. The measurements were carried out at 30° C. The sensors were equilibrated in a kinetic buffer solution for at least 10 minutes, then the baseline was recorded (60 s). Following the baseline recording, the sensors with immobilized Fabs were immersed into wells containing the analyte solution (recombinant human antigen BCMA-Avi-His-TEV-HSA), where the antigen-Fab complex was associated for 300 seconds. The human BCMA concentration was 391.2 nM (30 g/ml). The complex dissociation in a kinetic buffer solution was then detected for 600 seconds. To check the nonspecific interaction between the analyte and the sensors (negative control), we used sensors free of candidate Fabs. At the loading step, the negative control sensors were immersed into Fab-free supernatant, all other steps were similar to those used for the sensors loaded with Fab samples).

scFvs were measured on the Forte Bio Octert RED 384 system using SAX biosensors (ForteBio). For measurements, the human BCMA was biotinylated in a molar antigen/biotin ratio of 1:1.5. The interaction kinetics analysis was carried out at 30° C. The analysis was carried out using a kinetic buffer solution (4.3 mM Na₂HPO₄; 136.9 mM NaCl, 1.5 mM KH₂PO₄; 2.7 mM KCl; the volume fraction of the added Tween 20 was 0.1%; the mass fraction of the added BSA was 0.1%; pH 7.4). The biotinylated human BCMA was loaded onto sensors for 600 seconds, the concentration of the antigen solution in the kinetic buffer solution was 20 μg/ml. The baseline was recorded for 120 seconds. Following the baseline recording, the sensors with immobilized antigen were immersed into wells containing the analyte solution (candidate scFvs), where the antigen-scFv complex was associated for 150 seconds (for the association step, the scFv samples were diluted with a kinetic buffer solution to a concentration of 1200 nM). The antigen-scFv complex dissociation in a kinetic buffer solution was then detected for 600 seconds. The reference signal was recorded simultaneously with recording of candidate sensorgrams and was subtracted while processing the sensorgrams. The reference signal was the signal of a sensor immersed into the analyte-free kinetic buffer solution at the association step, all other steps were similar to sensors recording the test signal. Sensors not loaded with antigen were used to verify nonspecific interaction between the analyte (candidate scFvs) and sensors. At the loading step, the negative control sensors were immersed into antigen-free sodium kinetic buffer solution, and all other steps were analogous to those used for the antigen-loaded sensors).

Binding curves were analyzed using the Octet Data Analysis (Version 9.0) software and using the 1:1 interaction model. Analysis result for Fabs and scFvs of antibodies 01-001, 01-006, 01-016, 01-021, 01-022, 01-024, 01-025, 01-026, 01-027, 01-030, 01-033, 01-037 is shown in Table 1. These Fabs and scFvs were further used to create full-length antibodies.

TABLE 1
Results of interaction kinetics analysis for Fabs and
scFvs of antibodies 01-001, 01-006, 01-016, 01-021, 01-
022, 01-024, 01-025, 01-026, 01-027, 01-030, 01-033, 01-
037 with the human BCMA on ForteBio Octet RED384.

| | Re- | | | | |
|---|---|---|---|---|---|
| | sponse*, | KD, | Kon, | Kdis, | |
| Candidate | nm | M | 1/Ms | 1/s | R² |
| 01-001 (Fab) | 0.8314 | 104E−08 | 3.54E+04 | 3.67E−04 | 0.9919 |
| 01-006 (Fab) | 0.2955 | 5.04E−09 | 4.45E+04 | 2.24E−04 | 0.9718 |
| 01-016 (Fab) | 0.5322 | 8.46E−09 | 4.31E+04 | 3.65E−04 | 0.9876 |
| 01-021 (Fab) | 0.641 | 3.93E−09 | 6.78E+04 | 2.66E−04 | 0.9765 |
| 01-022 (scFv) | 0.1622 | 1.44E−08 | 7.11E+04 | 1.03E−03 | 0.9403 |
| 01-024 (scFv) | 0.1739 | 1.50E−09 | 3.80E+05 | 5.70E−04 | 0.8803 |
| 01-025 (scFv) | 0.1833 | 1.55E−08 | 8.26E+04 | 1.28E−03 | 0.9721 |
| 01-026 (scFv) | 0.1148 | 5.96E−08 | 4.17E+04 | 2.48E−03 | 0.9843 |
| 01-027 (scFv) | 0.1712 | 3.38E−09 | 2.84E+05 | 9.60E−04 | 0.8347 |
| 01-030 (scFv) | 0.1721 | 8.42E−09 | 1.12E+05 | 9.39E−04 | 0.9351 |
| 01-033 (scFv) | 0.1502 | 6.67E−09 | 1.12E+05 | 7.46E−04 | 0.9493 |
| 01-037 (scFv) | 0.1931 | 1.34E−08 | 7.07E+04 | 9.49E−04 | 0.9486 |
| Negative | 0.0368 | | | | |
| Control for | | | | | |
| Fab** | | | | | |
| Negative | ≤0.0272 | | | | |
| control for | | | | | |
| scFv*** | | | | | |

*Response - a signal level achieved at the end of association step;
**negative control for Fabs - signal of non-loaded FAB2G sensors while interacting with human BCMA;
***negative control for scFvs - signal of non-loaded SAX sensors with scFv sample solutions; measured for all samples in the scFv format, the highest value observed was 0.0272 nm.

### Example 6. Production of Genetic Constructs to Synthesize Full-Length Antibodies

Cloning was performed by the standard technique. We generated PCR products comprising variable domain genes of heavy and light chains of antibodies 01-001, 01-006, 01-016, 01-021, 01-022, 01-024, 01-025, 01-026, 01-027, 01-030, 01-033, 01-037 with primers comprising restriction sites. The heavy chain variable domain was cloned into the vector pEE-HCLALA IgG1 (FIG. 3) via Sal1/Nhe1 restriction sites. The light chain variable domain was cloned into the vector pEE-CK (FIG. 4) via Sal1/BsiW1 restriction sites. The resulting gene constructs were transferred for transient production of proteins in CHO-T cell line.

### Example 7. Production of Full-Length Antibodies

Full-length antibodies 01-001, 01-006, 01-016, 01-021, 01-022, 01-024, 01-025, 01-026, 01-027, 01-030, 01-033, 01-037 were produced in established cell line obtained from Chinese hamster ovary cells (CHO-T) using transient transfection in two replications. Suspension culturing was conducted in orbital shake bioreactors using serum-free media. For transient expression, cells at a concentration of 2-2.2×10⁶ cells/ml were transfected using linear polyethyleneimine. DNA/PEI ratio was 1:7. On day 10 of culturing, the cell suspension was centrifuged under 2000 g for 15 min and filtered through 0.22 μm filter.

Antibodies were purified by affinity chromatography columns using a robotic station. The column was equilibrated with a buffer containing 50 mM NaPB (sodium phosphate buffer), 150 mM NaCl (pH 7.5), the filtered culture liquid with antibodies was applied, the column was then washed with 8 volumes of a buffer containing 50 mM NaPB, 150 mM NaCl and 4 volumes of 50 mM NaPB (pH 7.5). Protein was eluted with 6 column volumes of a solution of 50 mM NaPB, 100 mM NaCl (pH 3). The resulting samples were neutralized with 25 μl of 1M phosphate buffer (pH 8).

Polyacrylamide gel electrophoresis and size-exclusion HPLC were used to monitor the purity of the candidates. Electrophoresis was performed in 7.5% polyacrylamide gel under denaturing non-reducing conditions. The protein purity was determined by the intensity of band staining at a protein load of 10 g per lane. Electrophoregrams are shown in FIG. 5 and FIG. 6. Size-exclusion HPLC was performed on a column in a mobile phase of 0.05M NaH2PO4, 0.3 M NaCl pH=7.0.

### Example 8. Determination of Affinity of Full-Length Antibodies 01-001, 01-006, 01-016, 01-021, 01-022, 01-024, 01-025, 01-026, 01-027, 01-030, 01-033, 01-037 to Human BCMA on Forte Bio

Experiments to study affinity of antibodies 01-001, 01-006, 01-016, 01-021, 01-022, 01-024, 01-025, 01-026, 01-027, 01-030, 01-033, 01-037 to human BCMA were conducted on the ForteBio Octet RED 384 system. Antibodies at a concentration of 20 μg/ml were immobilized onto the surface of Protein A biosensors (ForteBio). The analysis was carried out at 30° C. using a kinetic buffer solution (4.3 mM Na₂HPO₄; 136.9 mM NaCl, 1.5 mM KH₂PO₄; 2.7 mM KCl; the volume fraction of the added Tween 20 was 0.1%; the mass fraction of the added BSA was 0.1%; pH 7.4). After setting the baseline in the kinetic buffer solution, the sensors with immobilized antibodies were dipped into wells containing the analyte (BCMA) solution, where the association of the complex took place for 300 seconds. For each test antibody, a sensorgram for the human BCMA solution at a concentration of 10 μg/ml (130.4 nM) and a reference signal (reference sensorgram) of a BCMA-free kinetic buffer solution were recorded. The complex dissociation in buffer solution was then detected for 600 seconds. To verify nonspecific interaction between the analyte and the sensors (negative control), we used sensors not loaded with antibodies. At the loading step, the negative control sensors were immersed into antibody-free sodium acetate buffer, and all other steps were similar to those used for the sensors loaded with antibodies).

Binding curves, after subtracting a reference signal, were analyzed using the Octet Data Analysis (Version 9.0) software and using 1:1 interaction model (Table 2).

TABLE 2
Kinetic constants for antibodies/human BCMA interaction.

| Antibody | Response*, nm | KD, M | Kon, 1/Ms | Kdis, 1/s | R² |
|---|---|---|---|---|---|
| 01-001 | 1.0255 | 1.89E−09 | 8.37E+04 | 1.58E−04 | 0.9954 |
| 01-006 | 0.4332 | 5.16E−09 | 9.83E+04 | 5.08E−04 | 0.9851 |
| 01-016 | 1.0352 | 3.44E−09 | 8.05E+04 | 2.77E−04 | 0.9951 |
| 01-021 | 1.3325 | 2.15E−09 | 1.02E+05 | 2.18E−04 | 0.9924 |
| 01-022 | 0.6207 | 3.08E−09 | 1.05E+05 | 3.25E−04 | 0.9877 |
| 01-024 | 0.6046 | 5.34E−10 | 1.83E+05 | 9.77E−05 | 0.9973 |
| 01-025 | 0.5664 | 3.97E−09 | 8.15E+04 | 3.24E−04 | 0.9936 |
| 01-026 | 0.4853 | 6.20E−09 | 6.92E+04 | 4.29E−04 | 0.9936 |
| 01-027 | 0.6213 | 5.92E−10 | 1.80E+05 | 1.07E−04 | 0.9974 |
| 01-030 | 1.1529 | 1.82E−09 | 9.54E+04 | 1.73E−04 | 0.9946 |
| 01-033 | 1.0012 | 1.09E−09 | 9.95E+04 | 1.09E−04 | 0.9944 |
| 01-037 | 0.4618 | 2.91E−09 | 9.29E+04 | 2.70E−04 | 0.9842 |
| Negative | −0.0142 | | | | |
| control** | | | | | |

*Response - a signal level achieved at the end of association step;
**negative control - verification of nonspecific interaction between the analyte (human BCMA) and sensors not loaded with antibodies.

Thus, all test anti-BCMA antibodies specifically bind to the human BCMA and have high binding affinity parameters with respect to the human BCMA (Table 3).

### Example 13. Verification of Interactions Between Full-Sized Antibodies 01-001, 01-006, 01-016, 01-021, 01-022, 01-024, 01-025, 01-026, 01-027, 01-030, 01-033, 01-037 and Rhesus BCMA on the Forte Bio System

This experiment aimed to confirm interaction between antibodies 01-001, 01-006, 01-016, 01-021, 01-022, 01-024, 01-025, 01-026, 01-027, 01-030, 01-033, 01-037 and the rhesus BCMA. Interaction between antibodies and the rhesus BCMA was verified on the ForteBio Octet RED384 system using AR2G biosensors (ForteBio). The experiment consisted of the following steps: activating sensors, loading protein onto sensors, quenching unreacted activated groups, recording baselines, recording analyte association, recording dissociation. The measurements were carried out at 30° C. The sensors were activated in an aqueous solution comprising 20 mM EDC and 10 mM sNHS for 300 s. The rhesus BCMA was loaded onto the surface of biosensors in a 10 mM sodium-acetate buffer solution with pH 5.0 for 300 s. The concentration of loading protein was 10 μg/ml. Unreacted active centers on the sensor surfaces were quenched in 1M aqueous solution of ethanolamine with pH 8.5 for 300 s (pH value was adjusted by adding hydrochloric acid). To verify nonspecific interaction between the analyte and the sensors (negative control), we used an antigen-free sensor (at the loading step, the sensor was immersed in 10 mM sodium acetate buffer solution with pH 5.0; all other steps were analoguous to those used for the sensors loaded with antigen). The baseline and all subsequent steps of the experiment were carried out in a kinetic buffer solution (4.3 mM Na2HPO4; 136.9 mM NaCl, 1.5 mM KH2PO4; 2.7 mM KCl; the volume fraction of added Tween 20 was 0.100; the mass fraction of added BSA was 0.1%; pH 7.4). At the association step (step duration was 300 s), the rhesus BCMA-loaded sensors were immersed into wells with the analyte (test antibody) solution prepared in a kinetic buffer solution. The analyte concentration was 2.5 μg/ml. At the dissociation step, the sensors were immersed into wells with a kinetic buffer, where the baseline was recorded.

Binding curves were processed using the Octet Data Analysis (Version 9.0) software and using the 1:1 interaction model. The results of processing are shown in Table 4 (the value of kdis<1.0E-07 (l/s) and the corresponding thereto value of KD<1.0E-12 (M) means that, within the framework of the applied measurement and processing technique, the given sample shows exceeded limit of sensitivity of the dissociation rate constant for the model curve describing the sensorgram). The resulting kinetic constant values are of an estimate nature by virtue of the use of bivalent analyte (antibodies) and may only be used to compare samples. All anti-BCMA antibodies exhibit a signal of binding to the rhesus BCMA at the association step and a slow signal decay at the dissociation step, thus confirming high avidity while interaction with that antigen. Thus, all test anti-BCMA antibodies specifically bind to the rhesus BCMA (Table 3).

TABLE 3
Results of processing of sensorgrams
of antibody-rhesus BCMA binding.

| Antibody | Response*, nm | KD, M | Kon, 1/Ms | Kdis, 1/s | R² |
|---|---|---|---|---|---|
| 01-001 | 1.7867 | <1.0E−12 | 3.06E+05 | <1.0E−07 | 0.9595 |
| 01-006 | 1.4718 | 2.83E−11 | 4.85E+05 | 1.37E−05 | 0.9998 |
| 01-016 | 1.8205 | <1.0E−12 | 3.23E+05 | <1.0E−07 | 0.9611 |
| 01-021 | 1.8126 | <1.0E−12 | 3.54E+05 | <1.0E−07 | 0.9489 |
| 01-022 | 1.8809 | <1.0E−12 | 3.31E+05 | <1.0E−07 | 0.9534 |
| 01-024 | 1.8102 | 7.32E−12 | 4.96E+05 | 3.63E−06 | 0.9997 |
| 01-025 | 1.2699 | 4.02E−12 | 5.92E+05 | 2.38E−06 | 0.9992 |
| 01-026 | 1.4956 | <1.0E−12 | 4.91E+05 | <1.0E−07 | 0.9994 |
| 01-027 | 0.6259 | <1.0E−12 | 1.10E+06 | <1.0E−07 | 0.9897 |
| 01-030 | 1.1718 | <1.0E−12 | 8.22E+05 | <1.0E−07 | 0.9928 |
| 01-033 | 1.3062 | <1.0E−12 | 5.55E+05 | <1.0E−07 | 0.9993 |
| 01-037 | 1.5779 | <1.0E−12 | 5.27E+05 | <1.0E−07 | 0.9997 |
| Negative | ≤0.0150 | | | | |
| control** | | | | | |

The given values of the constants KD, Kon and Kdis can only be used to compare candidates, in view of the use of a bivalent analyte (antibody); the value of the constant Kdis <1.0E−07 (1/s) and the corresponding thereto value of KD <1.0E−12 (M) means that, within the framework of the applied measurement and processing technique, the given sample showed exceeded limit of sensitivity of the dissociation rate constant for the model curve describing the sensorgram;
*Response - a signal level achieved at the end of association step;
**negative control - verification of nonspecific interaction between the analyte (antibodies) and sensors not loaded with antigen; measured for all antibody samples, the highest value observed was 0.0150 nm.

### Example 14. Verification of Inhibition of Interaction Between APRIL and Human BCMA by Antibodies 01-001, 01-006, 01-016, 01-021, 01-022, 01-024, 01-025, 01-026, 01-027, 01-030, 01-033, 01-037 on Forte Bio System

The study was carried out on the ForteBio Octet RED 384 system using AR2G biosensors (ForteBio). The experiment consisted of the following main steps: loading the human APRIL/TNFSF13 antigen onto sensors, verification of binding of the APRIL/TNFSF13 antigen to a mixture of human BCMA and tested antibody (premix). A human BCMA solution free of antibodies served as a positive control. Inhibition of interaction was determining by comparing the premix signal (response) and positive control signal.

The sensors were activated in an aqueous solution comprising 20 mM EDC and 10 mM sNHS for 300 s. The APRIL/TNFSF13 antigen was loaded onto the surface of biosensors in a 10 mM sodium-acetate buffer with pH 5.0 for 900 s. The loading APRIL/TNFSF13 protein concentration was 10 μg/ml. To verify nonspecific interaction between the analyte and the sensors, we used an antigen-free sensor (at the loading step, the sensor was immersed in sodium acetate buffer with pH 5.0; all other steps were analogous to those used for the sensor loaded with antigen). Unreacted active centers on the sensor surfaces were quenched in 1M aqueous solution of ethanolamine with pH 8.5 (pH was adjusted by adding hydrochloric acid) for 300 s. All steps of the experiment, following the quenching step, were carried out in a kinetic buffer: (4.3 mM Na₂HPO₄; 136.9 mM NaCl, 1.5 mM KH₂PO₄; 2.7 mM KCl; the volume fraction of added Tween 20 was 0.1%; the mass fraction of added BSA (bovine serum albumin) was 0.1%; pH 7.4). At the association step (step duration was 300 s), sensors with loaded protein were immersed into wells with an analyte solution. A solution comprising 710 nM test antibody and 71 nM human BCMA in a kinetic buffer solution was used as an analyte at the association step. A solution (analyte) with human BCMA at a concentration of 71 nM served as a positive control. The measurements were carried out at 30° C. The reference sensors went through all the steps as the sensors used to record analyte sensorgrams did, with the exception of the association step—at the association step, the sensors were immersed in a kinetic buffer without analyte (the reference sensor signals were measured in parallel with the recording of the main sensorgrams). The reference signal was subtracted from the analyzed signal while processing the sensorgrams.

Sensorgrams were processed using ForteBio Octet Data Analysis in 9.0 software. The results of the experiment are shown in Table 4. Tested antibodies 01-001, 01-006, 01-016, 01-021, 01-022, 01-024, 01-025, 01-026, 01-027, 01-030, 01-033, 01-037 inhibit binding of APRIL/TNFSF13 loaded onto sensors to the human BCMA.

TABLE 4
Results of verification of inhibition of interaction
between APRIL/TNFSF13 and human BCMA by antibodies.

| | Protein loaded | Analyte (antibody + human | Response*, | |
|---|---|---|---|---|
| Antibodies | onto sensors | BCMA premix) | nm | Notes |
| 01-001 | APRIL/TNFSF13 | 710 nM 01-001 and 71 nM | −0.0081 | 01-001 inhibits |
| | | BCMA | | binding of BCMA to |
| | | | | APRIL/TNFSF13 |
| 01-006 | APRIL/TNFSF13 | 710 nM 01-006 and 71 nM | 0.0464 | 01-006 inhibits |
| | | BCMA | | binding of BCMA to |
| | | | | APRIL/TNFSF13 |
| 01-016 | APRIL/TNFSF13 | 710 nM 01-016 and 71 nM | −0.0062 | 01-016 inhibits |
| | | BCMA | | binding of BCMA to |
| | | | | APRIL/TNFSF13 |
| 01-021 | APRIL/TNFSF13 | 710 nM 01-021 and 71 nM | −0.0015 | 01-021 inhibits |
| | | BCMA | | binding of BCMA to |
| | | | | APRIL/TNFSF13 |
| 01-022 | APRIL/TNFSF13 | 710 nM 01-022 and 71 nM | 0.013 | 01-022 inhibits |
| | | BCMA | | binding of BCMA to |
| | | | | APRIL/TNFSF13 |
| 01-024 | APRIL/TNFSF13 | 710 nM 01-024 and 71 nM | 0.0151 | 01-024 inhibits |
| | | BCMA | | binding of BCMA to |
| | | | | APRIL/TNFSF13 |
| 01-025 | APRIL/TNFSF13 | 710 nM 01-025 and 71 nM | 0.0259 | 01-025 inhibits |
| | | BCMA | | binding of BCMA to |
| | | | | APRIL/TNFSF13 |
| 01-026 | APRIL/TNFSF13 | 710 nM 01-026 and 71 nM | 0.0111 | 01-026 inhibits |
| | | BCMA | | binding of BCMA to |
| | | | | APRIL/TNFSF13 |
| 01-027 | APRIL/TNFSF13 | 710 nM 01-027 and 71 nM | 0.017 | 01-027 inhibits |
| | | BCMA | | binding of BCMA to |
| | | | | APRIL/TNFSF13 |
| 01-030 | APRIL/TNFSF13 | 710 nM 01-030 and 71 nM | −0.0066 | 01-030 inhibits |
| | | BCMA | | binding of BCMA to |
| | | | | APRIL/TNFSF13 |
| 01-033 | APRIL/TNFSF13 | 710 nM 01-033 and 71 nM | −0.0078 | 01-033 inhibits |
| | | BCMA | | binding of BCMA to |
| | | | | APRIL/TNFSF13 |
| 01-037 | APRIL/TNFSF13 | 710 nM 01-037 and 71 nM | 0.0304 | 01-037 inhibits |
| | | BCMA | | binding of BCMA to |
| | | | | APRIL/TNFSF13 |
| Positive | APRIL/TNFSF13 | 71 nM BCMA | 0.6588 | Positive control, |
| control*** | | | | demonstrates binding |
| | | | | of BCMA to |
| | | | | APRIL/TNFSF13 |
| | | | | without inhibiting |
| | | | | antibody |
| Negative | Sensor was not | 71 nM BCMA | −0.0461 | Demonstrates absence |
| control** | loaded | | | of binding of the |
| | | | | analyte (71 nM |
| | | | | BCMA) to sensors |
| | | | | free of the |
| | | | | APRIL/TNFSF13 |
| | | | | antigen |

*Response - a signal level achieved at the end of association step;
**negative control - verification of nonspecific interaction between BCMA (antibodies) and sensors free of the antigen;
***positive control - binding signal (Response) of human BCMA to APRIL/TNFSF13 without inhibiting antibody.

### Example 15. Analysis of Binding of Anti-BCMA Antibodies 01-001, 01-006, 01-016, 01-021, 01-022, 01-024, 01-025, 01-026, 01-027, 01-030, 01-033, 01-037 to the Human BCMA on RPMI8226 Cells Surface by Flow Cytometry

Ability of anti-BCMA antibodies 01-001, 01-006, 01-016, 01-021, 01-022, 01-024, 01-025, 01-026, 01-027, 01-030, 01-033, 01-037 to bind to the human BCMA on cells surface was evaluated by flow cytometry on the RPMI8226 cell line (multiple myeloma) stably expressing the BCMA receptor on the surface thereof.

25,000 cells in a well were incubated in a staining buffer (PBS+NaN₃+5% BSA) for 30 minutes at +4° C. with serial dilutions of test antibodies. After that period of time, the cells were washed 2 times with a cold (+4° C.) staining buffer, and bound antibodies were detected by staining with anti-human Fc secondary antibodies labelled with Phycoerythrin for 30 minutes at +4° C. Wells without introduced antibodies and only with secondary antibodies were used as a control. After that period of time, the cells were washed 2 times with a cold (+4° C.) staining buffer and resuspended in 150 μl of a cold (+4° C.) staining buffer. The samples were then analyzed on a flow cytometer. We performed forward and side scatter (FSC and SSC) gating to distinguish the target cell population; thereafter, we performed forward scatter height and forward scatter area (FSC-H and FSC-A) gating to identify single (singlet) cells in the population.

Half-effective concentration EC₅₀ was calculated by plotting a four-parameter logistic regression model, the data are shown in Table 5.

TABLE 5
ED₅₀ of anti-BCMA antibodies while
binding to RPMI8226 cell surface.

| Antibody | EC₅₀ (μg/ml) |
|---|---|
| 01-001 | 0.59 |
| 01-006 | 0.45 |
| 01-016 | 1.05 |
| 01-021 | 0.49 |
| 01-022 | 0.76 |
| 01-024 | 0.22 |
| 01-025 | 0.70 |
| 01-026 | 1.75 |
| 01-027 | 0.25 |
| 01-030 | 0.33 |
| 01-033 | 0.39 |
| 01-037 | 0.79 |

Conclusion: the resulting data show that all tested antibodies bind to the BCMA target on multiple myeloma cell surface.

### Example 16. Analysis of Nonspecific Binding of Anti-BCMA Antibodies 01-001, 01-006, 01-016, 01-021, 01-022, 01-024, 01-025, 01-026, 01-027, 01-030, 01-033, 01-037 on BCMA-Negative K562 Cell Surface by Flow Cytometry

The presence of nonspecific binding of anti-BCMA antibodies 01-001, 01-006, 01-016, 01-021, 01-022, 01-024, 01-025, 01-026, 01-027, 01-030, 01-033, 01-037 was evaluated by flow cytometry on the K562 cell line (chronic myelogenous leukemia) which is characterized by the absence of BCMA expression on the surface thereof.

25,000 K562 cells in a well were incubated in a staining buffer (PBS+NaN₃+5% BSA) for 30 minutes at +4° C. with diluted test antibodies. After that period of time, the cells were washed 2 times with a cold (+4° C.) staining buffer, and bound antibodies were detected by staining with anti-human Fc secondary antibodies labelled with Phycoerythrin for 30 minutes at +4° C. Wells without introduced antibodies and only with secondary antibodies were used as a control. After that period of time, the cells were washed 2 times with a cold (+4° C.) staining buffer and resuspended in 150 μl of a cold (+4° C.) staining buffer. The samples were then analyzed on a flow cytometer. We performed forward and side scatter (FSC and SSC) gating to distinguish the target cell population; thereafter, we performed forward scatter height and forward scatter area (FSC-H and FSC-A) gating to identify single (singlet) cells in the population.

RPMI8226 was used as a positive control of cell-surface binding of test antibodies to BCMA. Sample preparation was carried out similarly to that described above for K562.

Nonspecific binding of antibodies was evaluated by comparing fluorescent signal levels from antibodies at the same concentration in the variants for K562 and RPMI8226 cell lines, the data are shown in Table 6.

TABLE 6
Data on cell-surface binding of anti-BCMA antibodies to K562
(BCMA-negative line) and RPMI8226 (BCMA-positive line).

Fluorescent signal level (sample/background)

| Antibody | RPMI8226 (BCMA⁺) | K562 (BCMA⁻) |
|---|---|---|
| 01-001 | 10.66 | 0.97 |
| 01-006 | 9.61 | 1.04 |
| 01-016 | 7.87 | 0.85 |
| 01-021 | 9.99 | 1.00 |
| 01-022 | 9.57 | 1.05 |
| 01-024 | 19.91 | 1.53 |
| 01-025 | 15.02 | 1.23 |
| 01-026 | 12.10 | 1.34 |
| 01-027 | 19.11 | 1.53 |
| 01-030 | 16.46 | 1.13 |
| 01-033 | 14.66 | 1.13 |
| 01-037 | 11.68 | 1.21 |

Conclusion: the results show that all tested antibodies do not exhibit nonspecific binding to the BCMA-negative cell line.

1. An isolated monoclonal antibody or antigen-binding fragment thereof that specifically binds to BCMA, including:
(a) a heavy chain variable domain comprising:
(i) CDR1 with the amino acid sequence SX₁X₂MS, where
X₁=S or G;
X₂=A, L or V;
(ii) CDR2 with the amino acid sequence X₃YNGGSDRAGX₄X₅DSVX₆G, where
X₃=G or C;
X₄=F or Y;
X₅=A or T;
X₆=E or K; and
(iii) CDR3 with the amino acid sequence of SEQ ID NO: 7;
and
(b) a light chain variable domain comprising:
(i) CDR1 with the amino acid sequence X₇GX₈X₉SNIGX₁₀X₁₁X₁₂X₁₃VX₁₄, where
X₇=S or T;
X₈=S, G or T;
X₉=S, T, I or R;
X₁₀=0 or A;
X₁₁=S, H, N, G or T;
X₁₂=N, R or Y;
X₁₃=T, A, I or D;
X₁₄=N or H;
(ii) CDR2 with the amino acid sequence X₁₅X₁₆X₁₇X₁₈RPS, where
X15=N, R, K, S or G;
X16=D, N, G, T or H;
X17=S, N or T;
X18=Q or N;
and
(iii) CDR3 with the amino acid sequence X₁₉X₂₀WDX₂₁X₂₂X₂₃X₂₄X₂₅WX₂₆, where
X₁₉=A or S;
X₂₀=A, S, T or V;
X₂₁=G, D, H or S;
X₂₂=S, D or R;
X₂₃=L or V;
X₂₄=N, T, R or S;
X₂₅=V, A, G or N;
X₂₆=M, V or L.

2. The isolated monoclonal antibody or antigen-binding fragment thereof according to claim 1, wherein the heavy chain variable domain comprises CDR1 with an amino acid sequence that is selected from the group: SEQ ID NO: 1, SEQ ID NO: 2 or SEQ ID NO: 3.

3. The isolated monoclonal antibody or antigen-binding fragment thereof according to claim 1, wherein the heavy chain variable domain comprises CDR2 with an amino acid sequence that is selected from the group: SEQ ID NO: 4, SEQ ID NO: 5 or SEQ ID NO: 6.

4. The isolated monoclonal antibody or antigen-binding fragment thereof according to claim 1, wherein the light chain variable domain comprises CDR1 with an amino acid sequence selected from the group: SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15.

5. The isolated monoclonal antibody or antigen-binding fragment thereof according to claim 1, wherein the light chain variable domain comprises CDR2 with an amino acid sequence selected from the group: SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 or SEQ ID NO: 24.

6. The isolated monoclonal antibody or antigen-binding fragment thereof according to claim 1, wherein the light chain variable domain comprises CDR3 with an amino acid sequence selected from the group: SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 or SEQ ID NO: 35.

7. The isolated monoclonal antibody or antigen-binding fragment thereof according to claim 1, including a heavy chain variable domain comprising:
CDR1 with an amino acid sequence that is selected from the group: SEQ ID NO: 1, SEQ ID NO: 2 or SEQ ID NO: 3;
CDR2 with an amino acid sequence that is selected from the group: SEQ ID NO: 4, SEQ ID NO: 5 or SEQ ID NO: 6;
and
CDR3 with the amino acid sequence of SEQ ID NO: 7.

8. The isolated monoclonal antibody or antigen-binding fragment thereof according to claim 7, including:
(i) a heavy chain variable domain comprising:
CDR1 with the amino acid sequence of SEQ ID NO: 1,
CDR2 with the amino acid sequence of SEQ ID NO: 4 and
CDR3 with the amino acid sequence of SEQ ID NO: 7;
(ii) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7;
(iii) a heavy chain variable domain comprising:
CDR1 with the amino acid sequence of SEQ ID NO: 3,
CDR2 with the amino acid sequence of SEQ ID NO: 4 and
CDR3 with the amino acid sequence of SEQ ID NO: 7;
(iv) a heavy chain variable domain comprising:
CDR1 with the amino acid sequence of SEQ ID NO: 2,
CDR2 with the amino acid sequence of SEQ ID NO: 4 and
CDR3 with the amino acid sequence of SEQ ID NO: 7;
or
(v) a heavy chain variable domain comprising:
CDR1 with the amino acid sequence of SEQ ID NO: 2,
CDR2 with the amino acid sequence of SEQ ID NO: 6 and
CDR3 with the amino acid sequence of SEQ ID NO: 7.

9. The isolated monoclonal antibody or antigen-binding fragment thereof according to claim 1, including a light chain variable domain comprising:
CDR1 with an amino acid sequence that is selected from the group: SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15;
CDR2 with an amino acid sequence that is selected from the group: SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 or SEQ ID NO: 24;
and
CDR3 with an amino acid sequence that is selected from the group: SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 or SEQ ID NO: 35.

10. The isolated monoclonal antibody or antigen-binding fragment thereof according to claim 9, including:
(i) a light chain variable domain comprising:
CDR1 with the amino acid sequence of SEQ ID NO: 8,
CDR2 with the amino acid sequence of SEQ ID NO: 16 and
CDR3 with the amino acid sequence of SEQ ID NO: 25;
(ii) a light chain variable domain comprising:
CDR1 with the amino acid sequence of SEQ ID NO: 9,
CDR2 with the amino acid sequence of SEQ ID NO: 17 and
CDR3 with the amino acid sequence of SEQ ID NO: 26;
(iii) a light chain variable domain comprising:
CDR1 with the amino acid sequence of SEQ ID NO: 10,
CDR2 with the amino acid sequence of SEQ ID NO: 18 and
CDR3 with the amino acid sequence of SEQ ID NO: 27;
(iv) a light chain variable domain comprising:
CDR1 with the amino acid sequence of SEQ ID NO: 11,
CDR2 with the amino acid sequence of SEQ ID NO: 19 and
CDR3 with the amino acid sequence of SEQ ID NO: 28;
(v) a light chain variable domain comprising:
CDR1 with the amino acid sequence of SEQ ID NO: 12,
CDR2 with the amino acid sequence of SEQ ID NO: 20 and
CDR3 with the amino acid sequence of SEQ ID NO: 29;
(vi) a light chain variable domain comprising:
CDR1 with the amino acid sequence of SEQ ID NO: 12,
CDR2 with the amino acid sequence of SEQ ID NO: 20 and
CDR3 with the amino acid sequence of SEQ ID NO: 30;
(vii) a light chain variable domain comprising:
CDR1 with the amino acid sequence of SEQ ID NO: 10,
CDR2 with the amino acid sequence of SEQ ID NO: 21 and
CDR3 with the amino acid sequence of SEQ ID NO: 31;
(viii) a light chain variable domain comprising:
CDR1 with the amino acid sequence of SEQ ID NO: 13,
CDR2 with the amino acid sequence of SEQ ID NO: 22 and
CDR3 with the amino acid sequence of SEQ ID NO: 32;
(ix) a light chain variable domain comprising:
CDR1 with the amino acid sequence of SEQ ID NO: 14,
CDR2 with the amino acid sequence of SEQ ID NO: 23 and
CDR3 with the amino acid sequence of SEQ ID NO: 33;
(x) a light chain variable domain comprising:
CDR1 with the amino acid sequence of SEQ ID NO: 8,
CDR2 with the amino acid sequence of SEQ ID NO: 24 and
CDR3 with the amino acid sequence of SEQ ID NO: 34;
or
(xi) a light chain variable domain comprising:
CDR1 with the amino acid sequence of SEQ ID NO: 15,
CDR2 with the amino acid sequence of SEQ ID NO: 17 and
CDR3 with the amino acid sequence of SEQ ID NO: 35.

11. The isolated monoclonal antibody or antigen-binding fragment thereof according to claim 1, including:
(a) a heavy chain variable domain comprising:
CDR1 with an amino acid sequence that is selected from the group: SEQ ID NO: 1, SEQ ID NO: 2 or SEQ ID NO: 3;
CDR2 with an amino acid sequence that is selected from the group: SEQ ID NO: 4, SEQ ID NO: 5 or SEQ ID NO: 6;
and
CDR3 with the amino acid sequence of SEQ ID NO: 7; and
(b) a light chain variable domain comprising:
CDR1 with an amino acid sequence that is selected from the group: SEQ ID NO: 8, SEQ ID NO: 9, SEQ ID NO: 10, SEQ ID NO: 11, SEQ ID NO: 12, SEQ ID NO: 13, SEQ ID NO: 14 or SEQ ID NO: 15;
CDR2 with an amino acid sequence that is selected from the group: SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 or SEQ ID NO: 24;
and
CDR3 with an amino acid sequence that is selected from the group: SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34 or SEQ ID NO: 35.

12. The isolated monoclonal antibody or antigen-binding fragment thereof according to claim 11, including:
(i) (a) a heavy chain variable domain comprising:
CDR1 with the amino acid sequence of SEQ ID NO: 1,
CDR2 with the amino acid sequence of SEQ ID NO: 4 and
CDR3 with the amino acid sequence of SEQ ID NO: 7; and
(b) a light chain variable domain comprising:
CDR1 with the amino acid sequence of SEQ ID NO: 8,
CDR2 with the amino acid sequence of SEQ ID NO: 16 and
CDR3 with the amino acid sequence of SEQ ID NO: 25;
(ii) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7; and
(b) a light chain variable domain comprising:
CDR1 with the amino acid sequence of SEQ ID NO: 9,
CDR2 with the amino acid sequence of SEQ ID NO: 17 and
CDR3 with the amino acid sequence of SEQ ID NO: 26;
(iii) (a) a heavy chain variable domain comprising:
CDR1 with the amino acid sequence of SEQ ID NO: 3,
CDR2 with the amino acid sequence of SEQ ID NO: 4 and
CDR3 with the amino acid sequence of SEQ ID NO: 7; and
(b) a light chain variable domain comprising:
CDR1 with the amino acid sequence of SEQ ID NO: 10,
CDR2 with the amino acid sequence of SEQ ID NO: 18 and
CDR3 with the amino acid sequence of SEQ ID NO: 27;
(iv) (a) a heavy chain variable domain comprising:
CDR1 with the amino acid sequence of SEQ ID NO: 2,
CDR2 with the amino acid sequence of SEQ ID NO: 4 and
CDR3 with the amino acid sequence of SEQ ID NO: 7; and
(b) a light chain variable domain comprising:
CDR1 with the amino acid sequence of SEQ ID NO: 11,
CDR2 with the amino acid sequence of SEQ ID NO: 19 and
CDR3 with the amino acid sequence of SEQ ID NO: 28;
(v) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7; and
(b) a light chain variable domain comprising:
CDR1 with the amino acid sequence of SEQ ID NO: 12,
CDR2 with the amino acid sequence of SEQ ID NO: 20 and
CDR3 with the amino acid sequence of SEQ ID NO: 29;
(vi) (a) a heavy chain variable domain comprising:
CDR1 with the amino acid sequence of SEQ ID NO: 2,
CDR2 with the amino acid sequence of SEQ ID NO: 6 and
CDR3 with the amino acid sequence of SEQ ID NO: 7; and
(b) a light chain variable domain comprising:
CDR1 with the amino acid sequence of SEQ ID NO: 12,
CDR2 with the amino acid sequence of SEQ ID NO: 20 and
CDR3 with the amino acid sequence of SEQ ID NO: 30;
(vii) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7; and
(b) a light chain variable domain comprising:
CDR1 with the amino acid sequence of SEQ ID NO: 10,
CDR2 with the amino acid sequence of SEQ ID NO: 21 and
CDR3 with the amino acid sequence of SEQ ID NO: 31;
(viii) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7, and
(b) a light chain variable domain comprising:
CDR1 with the amino acid sequence of SEQ ID NO: 13,
CDR2 with the amino acid sequence of SEQ ID NO: 22 and
CDR3 with the amino acid sequence of SEQ ID NO: 32;
(ix) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7; and
(b) a light chain variable domain comprising:
CDR1 with the amino acid sequence of SEQ ID NO: 12,
CDR2 with the amino acid sequence of SEQ ID NO: 20 and
CDR3 with the amino acid sequence of SEQ ID NO: 30;
(x) (a) a heavy chain variable domain comprising:
CDR1 with the amino acid sequence of SEQ ID NO: 2,
CDR2 with the amino acid sequence of SEQ ID NO: 4 and
CDR3 with the amino acid sequence of SEQ ID NO: 7; and
(b) a light chain variable domain comprising:
CDR1 with the amino acid sequence of SEQ ID NO: 14,
CDR2 with the amino acid sequence of SEQ ID NO: 23 and
CDR3 with the amino acid sequence of SEQ ID NO: 33;
(xi) (a) a heavy chain variable domain comprising:
CDR1 with the amino acid sequence of SEQ ID NO: 2,
CDR2 with the amino acid sequence of SEQ ID NO: 4 and
CDR3 with the amino acid sequence of SEQ ID NO: 7; and
(b) a light chain variable domain comprising:
CDR1 with the amino acid sequence of SEQ ID NO: 8,
CDR2 with the amino acid sequence of SEQ ID NO: 24 and
CDR3 with the amino acid sequence of SEQ ID NO: 34;
or
(xii) (a) a heavy chain variable domain comprising:
CDR1 with the amino acid sequence of SEQ ID NO: 2,
CDR2 with the amino acid sequence of SEQ ID NO: 5 and
CDR3 with the amino acid sequence of SEQ ID NO: 7; and
(b) a light chain variable domain comprising:
CDR1 with the amino acid sequence of SEQ ID NO: 15,
CDR2 with the amino acid sequence of SEQ ID NO: 17 and
CDR3 with the amino acid sequence of SEQ ID NO: 35.

13. The isolated monoclonal antibody or antigen-binding fragment thereof according to claim 1, wherein the heavy chain variable domain comprises an amino acid sequence selected from the group: SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43.

14. The isolated monoclonal antibody or antigen-binding fragment thereof according to claim 1, wherein the light chain variable domain comprises an amino acid sequence selected from the group: SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53 or SEQ ID NO: 54.

15. The isolated monoclonal antibody or antigen-binding fragment thereof according to claim 1, wherein:
(a) the heavy chain variable domain comprises an amino acid sequence selected from the group: SEQ ID NO: 36, SEQ ID NO: 37, SEQ ID NO: 38, SEQ ID NO: 39, SEQ ID NO: 40, SEQ ID NO: 41, SEQ ID NO: 42 or SEQ ID NO: 43; and
(b) the light chain variable domain comprises an amino acid sequence selected from the group: SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53 or SEQ ID NO: 54.

16. The isolated monoclonal antibody or antigen-binding fragment thereof according to claim 15, wherein:
(i) (a) the heavy chain variable domain comprises the amino acid sequence of SEQ ID NO: 36 and
(b) the light chain variable domain comprises the amino acid sequence of SEQ ID NO: 44;
(ii) (a) the heavy chain variable domain comprises the amino acid sequence of SEQ ID NO: 37 and
(b) the light chain variable domain comprises the amino acid sequence of SEQ ID NO: 45;
(iii) (a) the heavy chain variable domain comprises the amino acid sequence of SEQ ID NO: 38 and
(b) the light chain variable domain comprises the amino acid sequence of SEQ ID NO: 46;
(iv) (a) the heavy chain variable domain comprises the amino acid sequence of SEQ ID NO: 39 and
(b) the light chain variable domain comprises the amino acid sequence of SEQ ID NO: 47;
(v) (a) the heavy chain variable domain comprises the amino acid sequence of SEQ ID NO: 40 and
(b) the light chain variable domain comprises the amino acid sequence of SEQ ID NO: 48;
(vi) (a) the heavy chain variable domain comprises the amino acid sequence of SEQ ID NO: 41 and
(b) the light chain variable domain comprises the amino acid sequence of SEQ ID NO: 49;
(vii) (a) the heavy chain variable domain comprises the amino acid sequence of SEQ ID NO: 40 and
(b) the light chain variable domain comprises the amino acid sequence of SEQ ID NO: 50;
(viii) (a) the heavy chain variable domain comprises the amino acid sequence of SEQ ID NO: 40 and
(b) the light chain variable domain comprises the amino acid sequence of SEQ ID NO: 51;
(ix) (a) the heavy chain variable domain comprises the amino acid sequence of SEQ ID NO: 40 and
(b) the light chain variable domain comprises the amino acid sequence of SEQ ID NO: 49;
(x) (a) the heavy chain variable domain comprises the amino acid sequence of SEQ ID NO: 42 and
(b) the light chain variable domain comprises the amino acid sequence of SEQ ID NO: 52;
(xi) (a) the heavy chain variable domain comprises the amino acid sequence of SEQ ID NO: 43 and
(b) the light chain variable domain comprises the amino acid sequence of SEQ ID NO: 53;
or
(xii) (a) the heavy chain variable domain comprises the amino acid sequence of SEQ ID NO: 40 and
(b) the light chain variable domain comprises the amino acid sequence of SEQ ID NO: 54.

17. The isolated monoclonal antibody according to any one of claims 1 to 16, wherein the antibody that specifically binds to BCMA is a full-length IgG antibody.

18. The isolated monoclonal antibody according to claim 17, wherein the full-length IgG antibody is of human IgG1, IgG2, IgG3 or IgG4 isotype.

19. The isolated monoclonal antibody according to claim 18, wherein the full-length IgG antibody is of human IgG1 isotype.

20. The isolated monoclonal antibody according to claim 19, wherein the antibody comprises mutations L234A and L235A according to the EU numbering of amino acids in the CH2 region.

21. The isolated monoclonal antibody according to claim 1, including a heavy chain comprising an amino acid sequence selected from the group: SEQ ID NO: 55, SEQ ID NO: 56, SEQ ID NO: 57, SEQ ID NO: 58, SEQ ID NO: 59, SEQ ID NO: 60, SEQ ID NO: 61 or SEQ ID NO: 62.

22. The isolated monoclonal antibody according to claim 1, including a light chain comprising an amino acid sequence selected from the group: SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72 or SEQ ID NO: 73.

23. The isolated monoclonal antibody according to claim 1, including:
(i) (a) a heavy chain comprising an amino acid sequence that is selected from the group: SEQ ID NO: 55, SEQ ID NO: 56, SEQ ID NO: 57, SEQ ID NO: 58, SEQ ID NO: 59, SEQ ID NO: 60, SEQ ID NO: 61 or SEQ ID NO: 62, and
(b) a light chain comprising an amino acid sequence that is selected from the group: SEQ ID NO: 63, SEQ ID NO: 64, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72 or SEQ ID NO: 73.

24. The isolated monoclonal antibody according to claim 23, including:
(i) (a) a heavy chain comprising the amino acid sequence of SEQ ID NO: 55, and
(b) a light chain comprising the amino acid sequence of SEQ ID NO: 63;
(ii) (a) a heavy chain comprising the amino acid sequence of SEQ ID NO: 56, and
(b) a light chain comprising the amino acid sequence of SEQ ID NO: 64;
(iii) (a) a heavy chain comprising the amino acid sequence of SEQ ID NO: 57, and
(b) a light chain comprising the amino acid sequence of SEQ ID NO: 65;
(iv) (a) a heavy chain comprising the amino acid sequence of SEQ ID NO: 58, and
(b) a light chain comprising the amino acid sequence of SEQ ID NO: 66;
(v) (a) a heavy chain comprising the amino acid sequence of SEQ ID NO: 59, and
(b) a light chain comprising the amino acid sequence of SEQ ID NO: 67;
(vi) (a) a heavy chain comprising the amino acid sequence of SEQ ID NO: 60, and
(b) a light chain comprising the amino acid sequence of SEQ ID NO: 68;
(vii) (a) a heavy chain comprising the amino acid sequence of SEQ ID NO: 59, and
(b) a light chain comprising the amino acid sequence of SEQ ID NO: 69;
(viii) (a) a heavy chain comprising the amino acid sequence of SEQ ID NO: 59, and
(b) a light chain comprising the amino acid sequence of SEQ ID NO: 70;
(ix) (a) a heavy chain comprising the amino acid sequence of SEQ ID NO: 59, and
(b) a light chain comprising the amino acid sequence of SEQ ID NO: 68,
(x) (a) a heavy chain comprising the amino acid sequence of SEQ ID NO: 61, and
(b) a light chain comprising the amino acid sequence of SEQ ID NO: 71;
(xi) (a) a heavy chain comprising the amino acid sequence of SEQ ID NO: 62, and
(b) a light chain comprising the amino acid sequence of SEQ ID NO: 72;
or
(xii) (a) a heavy chain comprising the amino acid sequence of SEQ ID NO: 59, and
(b) a light chain comprising the amino acid sequence of SEQ ID NO: 73.

25. The isolated nucleic acid that encodes the antibody or antigen-binding fragment thereof according to any one of claims 1 to 24.

26. The isolated nucleic acid according to claim 25, wherein the nucleic acid is DNA.

27. An expression vector comprising the nucleic acid according to any one of claims 25 to 26.

28. A method for producing a host cell to produce the antibody or antigen-binding fragment thereof according to any one of claims 1 to 24, including cell transformation by the vector according to claim 27.

29. A host cell for producing the antibody or antigen-binding fragment thereof according to any one of claims 1 to 24, comprising the nucleic acid according to any one of claims 25 to 26.

30. A method for producing the antibody or antigen-binding fragment thereof according to any one of claims 1 to 24, comprising culturing the host cell according to claim 29 in a growth medium under conditions sufficient to produce said antibody, if necessary, followed by isolation and purification of the resulting antibody.

31. A pharmaceutical composition for treating a BCMA-mediated disease or disorder comprising the antibody or antigen-binding fragment thereof according to any one of claims 1 to 24 in a therapeutically effective amount in combination with one or more pharmaceutically acceptable excipients.

32. The pharmaceutical composition according to claim 31, wherein the BCMA-mediated disease or disorder is selected from the group: multiple myeloma, chronic lymphocytic leukemia, B-cell acute lymphoblastic leukemia, non-Hodgkin's lymphoma, Hodgkin's lymphoma.

33. A pharmaceutical composition for treating a BCMA-mediated disease or disorder, comprising the antibody or antigen-binding fragment thereof according to any one of claims 1 to 24 and at least one other therapeutically active compound.

34. The pharmaceutical composition according to claim 33, wherein the BCMA-mediated disease or disorder is selected from the group: multiple myeloma, chronic lymphocytic leukemia, B-cell acute lymphoblastic leukemia, non-Hodgkin's lymphoma, Hodgkin's lymphoma.

35. The pharmaceutical composition according to any one of claims 33 to 34, wherein the other therapeutically active compound is an antibody, a small molecule, a hormone therapy agent or combination thereof.

36. A method for treating a BCMA-mediated disease or disorder, including administering to a subject in need of such treatment the antibody or antigen-binding fragment thereof according to any one of claims 1 to 24 or the pharmaceutical composition according to any one of claims 31 to 35 in a therapeutically effective amount.

37. The method for treating a disease or disorder according to claim 36, wherein the BCMA-mediated disease or disorder is selected from the group: multiple myeloma, chronic lymphocytic leukemia, B-cell acute lymphoblastic leukemia, non-Hodgkin's lymphoma, Hodgkin's lymphoma.

38. A method for treating a BCMA-mediated disease or disorder, including administering to a subject in need of such treatment the antibody or antigen-binding fragment thereof according to any one of claims 1 to 24 and at least one other therapeutically active compound in a therapeutically effective amount.

39. The method for treating a disease or disorder according to claim 38, wherein the BCMA-mediated disease or disorder is selected from the group: multiple myeloma, chronic lymphocytic leukemia, B-cell acute lymphoblastic leukemia, non-Hodgkin's lymphoma, Hodgkin's lymphoma.

40. The method for treating a disease or disorder according to any one of claims 38 to 39, wherein the other therapeutically active compound is an antibody, a small molecule, a hormone therapy agent or combination thereof.

41. Use of the antibody or antigen-binding fragment thereof according to any one of claims 1 to 24 or the pharmaceutical composition according to any one of claims 31 to 35 for treating a BCMA-mediated disease or disorder in a subject in need of such treatment.

42. The use according to claim 41, wherein the BCMA-mediated disease or disorder is selected from the group: multiple myeloma, chronic lymphocytic leukemia, B-cell acute lymphoblastic leukemia, non-Hodgkin's lymphoma, Hodgkin's lymphoma.

43. The use of the antibody or antigen-binding fragment thereof according to any one of claims 1 to 24 and at least one other therapeutically active compound for treating a BCMA-mediated disease or disorder.

44. The use according to claim 43, wherein the BCMA-mediated disease or disorder is selected from the group: multiple myeloma, chronic lymphocytic leukemia, B-cell acute lymphoblastic leukemia, non-Hodgkin's lymphoma, Hodgkin's lymphoma.

45. The use according to claim 43, wherein the other therapeutically active compound is a small molecule, hormone therapy agent, or any combination thereof.
